(19) United States
(12) Patent Application Publication
(10) Pub. No.: US 2009/0230823 A1
(43) Pub. Date: Sep. 17, 2009

(54) Operation of patterned ultrasonic transducers

(76) Inventors: Leonid Kushculey, Rehovot (IL); Vladimir Goland, Ashdod (IL)

Correspondence Address: EMPK & Shiloh, LLP;c/o Landon IP, Inc., 1700 Diagonal Road, Suite 450, Alexandria, VA, 22314, US

(21) Appl. No.: 12/081,379

(22) Filed: Apr. 15, 2008

Related U.S. Application Data
(60) Provisional application No. 61/064,581, filed on Mar. 13, 2008.

(51) Int. Cl. H01L 41/047; A61N 7/00

(52) U.S. Cl. 310/366; 601/2

(57) ABSTRACT

There is provided a transducer array comprising at least one unitary piece of piezoelectric material having first and second opposing surfaces; and a conductive layer on each of said first and second opposing surfaces, wherein at least one of said conductive layers is divided up into a plurality of electrode elements, and wherein said electrode elements, independently, are adapted to receive excitation energy of at least one of a predetermined amplitude and phase.

### RELATED APPLICATIONS

This application claims the benefit of U.S. Provisional Patent Application No. 61/064,581, filed Mar. 13, 2008, which is incorporated herein by reference.

### FIELD OF THE DISCLOSURE

The present disclosure relates to the field of the use of multiple element transducers for ultrasonic treatment of tissue.

### BACKGROUND

Ultrasound is widely used in medicine for diagnostic and therapeutic applications. Therapeutic ultrasound may induce a vast range of biological effects at very different exposure levels. At low levels, beneficial, reversible cellular effects can be produced, whereas at higher intensities, instantaneous cell death can occur. Accordingly ultrasound therapies can be broadly divided into two groups: “high” power and “low” power therapies. At the one end of the spectrum, high power applications include high intensity focused ultrasound (HIFU) and lithotripsy, while at the other end, low power applications comprise sonophoresis, sonoporation, gene therapy, bone healing, and the like.

A popular area in the field of aesthetic medicine is the removal of subcutaneous fat and the reduction of the volume of adipose tissue, resulting in the reshaping of body parts, frequently referred to as “body contouring”. One such technique is a non-invasive ultrasound-based procedure for fat and adipose tissue removal. The treatment is based on the application of focused therapeutic ultrasound that selectively targets and disrupts fat cells without damaging neighboring structures. This may be achieved by, for example, a device, such as a transducer, that delivers focused ultrasound energy to the subcutaneous fat layer. Specific, pre-set ultrasound parameters are used in an attempt to ensure that only the fat cells within the treatment area are targeted and that neighboring structures such as blood vessels, nerves and connective tissue remain intact.

Focused high intensity acoustic energy is also used for therapeutic treatment of various medical conditions, including the non-invasive destruction of tumerous growths by tissue ablation or destruction.

For such medical and cosmetic purposes, it is often desirable to be able to focus the ultrasonic output of the transducer. To achieve this, transducers are often comprised of a cup-shaped piezoelectric ceramic shell with conductive layers forming a pair of electrodes covering the convex outside and concave inside of the piezoelectric shell. Typically, the transducers have the shape of a segment of a sphere, with the “open end” positioned toward the subject being treated.

The transducer is excited to vibrate and generate ultrasound by pulsing it using a high frequency power supply generally operating at a resonant frequency of vibration of the piezoelectric material.

Such a spherical transducer exhibits an “axial focal pattern”. This is an ellipsoidal pattern having a relatively small cross section and a relatively longer axis coincident with a “longitudinal” axis of the transducer, for example, a line through the center of rotation of the transducer perpendicular to the equatorial plane. However, since the dimensions of the focused volume are small, being of the order of 1.5 mm in radius for 1 MHz ultrasound emission, in order to treat relatively large volumes of tissue, it would be generally advantageous to modify the focal pattern so that it is spread laterally and longitudinally.

Furthermore, since cosmetic treatments in particular, and efficient apparatus utilization in general, are sensitive to the time taken to perform the procedure, methods whereby a singly focused region is moved over the subject's body are unattractive commercially, and better efficacy of such treatments would be desirable.

Other types of transducers are planar in shape, generating a sheet of energy at the target plane, but the focusing power of such transducers is limited. Such planar transducers may also incorporate an acoustic lens to focus energy to a desired location.

Transducers which emit ultrasound in a single focused beam have limitations, such as being single-frequencied, which can be overcome by the use of multiple segment transducers. Such prior art, multiple segment transducers are generally constructed of a number of separate ceramic piezoelectric elements glued together, or epoxy embedded, in order to produce a single integrated head. However, transducers produced by such methods are generally costly to manufacture because of the labor intensive process of manufacture, and are often unreliable because of the susceptibility of the adhesive or epoxy matrix to loosen, degrade, or otherwise interfere with the transducers under the effects of high intensity ultrasound.

### SUMMARY

The present disclosure seeks to provide new uses for multiply segmented transducer heads, especially as applied to increasing the efficacy of fat removal. The methods are generally enabled by use of a segmented transducer structure, in which a single, unitary sample of piezoelectric material having two opposite surfaces is induced to operate as if it were composed of a plurality of smaller individual transducer segments, by means of electrically separate electrode elements applied to at least one surface of the two opposite surfaces, wherein each electrode element is associated with a transducer segment. The application of the electrode elements to the at least one surface can be performed either by dividing up a continuous electrode preformed on a surface of the material, generally by scribing or cutting the surface, or by applying a coating to the surface in the form of electrically separate electrode elements. Each of the separate electrode elements can then be activated separately by its own applied high frequency voltage, applied between the segment and an electrode on the opposing surface of the sample. Such a multi-element transducer has a structure which is simpler to construct than an adhesively assembled multi-element transducer, and which is also generally more reliable. Furthermore, the individual transducer segments generally operate independently of each other, and, except for some small effects on close neighbors, do not mutually interfere, thus enabling additive combinations of their outputs to be synthesized by appropriate excitation of the associated electrodes. According to some embodiments of the present disclosure, the single component base transducer can be constructed to have separate regions of different vibrational frequency when excited, and the electrodes arranged to overlie these separate regions, such that a multiple frequency ultrasound emission can be provided by exciting the separate electrode regions.

Different transducer segments, or different groups of transducer segments, or different samples of the piezoelectric material may be excited with high frequency voltages at different amplitudes and having different mutual phases, such that these segments or groups of segments, or samples, act as a phased array. Selection of the applied amplitudes and phases causes the transducer to emit ultrasound in a predetermined direction, or to sweep the emitted ultrasound through a predetermined range of directions. When used for treating a subject, this enables a larger region to be treated without moving the transducer head, so reducing the treatment time. Additionally, the focal position and size can be more accurately controlled, thus enabling safer operation in proximity to sensitive areas.

According to further embodiments of the disclosure, the excitation applied to the different segments or groups of segments need not have specific phase relationships, such that they do not have the characteristics of a phased array, but are rather operated either sequentially or additively to generate predetermined spatial effects on the tissue being treated.

Different modes of operation of arrays of patterned ultrasound transducers, according to different embodiments of the present disclosure, whether phase controlled or not, may thus be used for a number of different special effects for increasing the efficacy or specificity of ultrasound treatment of bodily tissues. Among the parameters used for these effects are the placement of the excited segments or groups of segments, the phase relationships between the exciting fields applied to the segments or groups of segments, the vibrational frequencies emitted by the segments or groups of segments, and the harmonic content preferentially generated by the segments or groups of segments. Among the local effects which can be emphasized or minimized by use of such arrays of patterned ultrasound transducers, are included the treatment of a target region substantially larger than the focal zone of the emitted energy, without the need to move the transducer head; the selective impingement of the energy on target tissues, to the exclusion of significant effects on neighboring non-target tissue; the selective treatment of some types of cells in the target area to the exclusion of significant effects on different, non-targeted cell types within the target area, by use of selective levels of energy on a target; and the control of the ratio between the main lobe and the side lobes of a propagation pattern, to control the physiological effect of the impinging propagated beam.

There is therefore provided, according to an embodiment of the disclosure, a transducer array comprising at least one unitary piece of piezoelectric material having first and second opposing surfaces; and a conductive layer on each of said first and second opposing surfaces, wherein at least one of said conducting layers is divided up into a plurality of electrode elements, and wherein said electrode elements, independently, are adapted to receive excitation energy of at least one of a predetermined amplitude and phase.

There is further provided, according to an embodiment of the disclosure, a transducer array comprising at least one unitary element of piezoelectric material operative as a plurality of individual transducer segments by virtue of a plurality of electrode elements, said plurality of electrode elements being formed as a segmented conductive layer on at least one surface of said at least one unitary element of piezoelectric material, each segment of said conductive layer defining an individual transducer segment; and driving circuitry for supplying high frequency voltages to at least some of said electrode elements, such that said individual transducer segments associated with said at least some electrode elements emit ultrasound energy, wherein said driving circuitry varies at least one of an amplitude and a phase of said high frequency voltages applied to different ones of said at least some electrode elements, so as to affect propagation of said emitted ultrasound energy.

In some embodiments, the ultrasound energy emitted from said transducer array is influenced by at least one of the amplitudes and phases of the excitation energy received by the electrode elements.

In some embodiments, said phases are adapted to be shifted such that said ultrasound energy emitted from said transducer array is directed at an angle in accordance with the shift of said phases.

In some embodiments, the shift of said phases is adapted to vary as a function of time, such that said ultrasound energy executes a sweeping action in accordance with said variation of said phase shift.

In some embodiments, at least one of said amplitude and said phase is adapted to vary, such that a focal position of said ultrasound energy emitted from said transducer array is controlled.

In some embodiments, at least one of said amplitude and said phase is adapted to vary, such that a profile of said ultrasound energy emitted from said transducer array is amended.

In some embodiments, amendment of said profile of said ultrasound energy emitted from said transducer array changes a mutual relationship between a main lobes and side lobes of said profile.

In some embodiments, for a given sweep range, the mutual relationship between the main lobes and the side lobes of said propagation is controlled by changing said amplitude as a function of a position of said ultrasound energy emitted from said transducer array in said sweep range.

In some embodiments, said control of the focal position enables an increase in a target volume that said ultrasound emission can treat without motion of said transducer array.

In some embodiments, said control of the focal position increases an accuracy of the focal position of said ultrasound emission, such that impingement on undesired regions is reduced.

In some embodiments, said at least one of said amplitude and phase is adapted to vary so as to generate, within a target area, at least two focused regions from different regions of said array.

In some embodiments, said at least two focused regions are directed to fall essentially on a same position within said target area such that an intensity of said ultrasound in said target area is increased.

In some embodiments, said at least two focused regions are directed to fall close to each other within said target area such that a volume of said target area is increased.

In some embodiments, said at least one of the amplitude and phase is adapted to vary so as to control a type of interaction of said ultrasound energy on a tissue of a subject.

There is further provided, according to an embodiment of the disclosure, a method of generating ultrasound energy, comprising providing at least one unitary element of piezoelectric material having conductive layers on its first and second surfaces, at least one of said conductive layers being a segmented layer comprising a plurality of electrode elements, each of said electrode elements defining a segmental transducer; exciting at least some of said electrode elements with high frequency voltages such that their associated segmental transducers emit ultrasound energy; and varying at least one of the amplitude and phase of said high frequency voltages applied to different ones of at least some of said electrode elements, so as to influence the propagation of said ultrasound energy emitted from said transducer array.

There is further provided, according to an embodiment of the disclosure, a method of generating ultrasound energy, comprising providing at least one unitary element of piezoelectric material operative as a plurality of individual transducer segments by exciting a plurality of electrode elements, said plurality of electrode elements being formed as a segmented conductive layer on a surface of said at least one unitary element of piezoelectric material, each segment of said conductive layer defining an individual transducer segment; applying high frequency voltages to at least some of said electrode elements, such that said individual transducer segments associated with said at least some electrode elements emit ultrasound energy; and varying at least one of the amplitude and phase of said high frequency voltages applied to different ones of said at least some electrode elements so as to affect the propagation of said emitted ultrasound energy.

In some embodiments, a phase shift applied to different ones of said at least some electrode elements is varied as a function of time, such that said ultrasound energy executes a sweep in accordance with the variation of said phase shift.

In some embodiments, at least one of said amplitude and said phase of said high frequency voltages applied to different ones of said at least some electrode elements is varied, such that a profile of said ultrasound energy emitted from said transducer array is amended.

In some embodiments, at least one of said amplitude and said phase of said high frequency voltages applied to different ones of said at least some electrode elements is varied such that a position of focus of said ultrasound energy emitted from said transducer array is controlled.

In some embodiments, amendment of said profile of said ultrasound energy emitted from said transducer array changes a mutual relationship between main lobes and side lobes of said profile.

In some embodiments, for a given sweep range, the mutual relationship between the main lobes and side lobes of said profile is controlled by changing said amplitude of said high frequency voltages as a function of the position of said emitted ultrasound energy in said sweep range.

In some embodiments, said control of the position of focus enables an increase in a target volume that said ultrasound emission can treat without motion of said transducer array.

In some embodiments, said control of the position of focus increases accuracy of the focal position of said ultrasound emission, such that impingement on undesired regions is reduced.

In some embodiments, said at least one of said amplitude and phase applied to different ones of said at least some electrode elements is varied so as to generate within a target area at least two focused regions from different regions of said array.

In some embodiments, said at least two focused regions are directed to fall essentially on the same position within said target area such that intensity of said ultrasound in said target area is increased.

In some embodiments, said at least two focused regions are directed to fall close to each other within said target area such that the volume of said target area is increased.

In some embodiments, said at least one of the amplitude and phase of said high frequency voltages applied to different ones of said at least some electrode elements is varied so as to control the type of interaction of said ultrasound energy on a tissue of a subject.

There is further provided, according to an embodiment of the disclosure, a method of moving ultrasound energy through a target volume, comprising providing at least one unitary element of piezoelectric material having conductive layers on its surfaces, at least one of said conductive layers being a segmented layer comprising a plurality of electrode elements, each of said electrode elements defining a segmental transducer; positioning said at least one unitary element of piezoelectric material in proximity to said target area; exciting at least some of said segments with high frequency voltages such that their associated segmental transducer emit ultrasound energy; and varying the phase of said high frequency voltages applied to different ones of at least some of said segments such that said ultrasound moves through said target volume.

### BRIEF DESCRIPTION OF THE FIGURES

The present disclosure will be understood and appreciated more fully from the following detailed description, taken in conjunction with the drawings in which:

FIGS. 1A shows schematically a cross sectional view of a prior art ultrasonic dome shaped focusing piezoelectric transducer being used to provide high intensity focused ultrasound (HIFU);

FIG. 1B schematically illustrates a spherical segment transducer;

FIGS. 2A and 2B illustrate schematically embodiments of a multiple transducer head, comprising a single spherical ceramic element having a segmented electrode;

FIGS. 3A to 3F show schematically various differently shaped transducer heads, each constructed using a multi-element electrode on a unitary ceramic base transducer; FIG. 3E shows such a head made up of two pieces of ceramic;

FIGS. 4A to 4B illustrate schematically transducer heads constructed to operate at multiple frequencies by means of regions of different thickness, according to some embodiments;

FIG. 5 shows schematically a single element transducer constructed to operate at multiple frequencies;

FIGS. 6A to 6C illustrate schematically possible arrangements of segmented electrode transducer elements with a small number of segments;

FIGS. 7A to 7C illustrate schematically additional possible arrangements of arrays of separate transducer elements, both symmetric and non-symmetric:

FIG. 8 illustrates schematically the method of phased array beam steering using a flat array of transducers, such as that shown in the embodiment of FIG. 3C;

FIG. 9 illustrates schematically the effect of the application of the phased array beam steering technique shown in FIG. 8, to a cap-shaped segmented transducer, such as that shown in the embodiment of FIG. 2A;

FIG. 10 shows an embodiment in which an array of transducers fired sequentially may be used to increase either or both of the volume coverage and the energy density obtainable from a single transducer head without moving the head;

FIG. 11 shows an exemplary schematic spherical cap-shaped unitary transducer, according to some embodiments;

FIG. 12 illustrates a graph of intensity profile of the ultrasound energy impinging on target, according to some embodiments;

FIG. 13 schematically illustrates an array of segmented transducers, according to some embodiments;

FIG. 14 illustrates hydrophone measurement of acoustic field distribution in the focal plane of a transducer;

FIG. 15 illustrates an ultrasound image showing a cavitation event produced by a transducer in hydrogel;

FIG. 16 illustrates a graph of the temperature variations with time in the focus;

FIG. 17 illustrates a graph of the radial temperature increase distribution in the focal plane;

FIGS. 18A-B show a pictorial macroscopic histological evaluation of a swine adipose tissue;

FIGS. 19A-B show pictorial LDH staining evaluation of a swine adipose tissue;

FIGS. 20A-F show pictorial microscopic histological evaluation of swine tissues;

FIG. 21 illustrates a graph of mean circumference reduction over time, of a single-treatment clinical trial;

FIG. 22 illustrates a graph of change in weight over time, of a single-treatment clinical trial;

FIG. 23 illustrates a flow chart of a method for generating focused ultrasound energy; and

FIG. 24 illustrates a body contouring treatment of a patient.

### DETAILED DESCRIPTION

Glossary

Below is presented a list of terms related to ultrasound equipment and ultrasonic output measurements which are used throughout the following disclosure:

As referred to herein, the term “Beam Axis” relates to a straight line joining the points of the maximum “Pulse Intensity Integral” measured at several different distances in the far field. This line is to be extended back to a transducer surface.

As referred to herein, the term “Beam Cross-Sectional Area” relates to the area on the surface of the plane perpendicular to the “Beam Axis” consisting of all points where the acoustic pressure is greater than 50% of the maximum acoustic pressure in the plane.

As referred to herein, the term “Duty Cycle (DC)” relates to the ratio of “Pulse Duration” to “Pulse Repetition Period”.

As referred to herein, the term “Focal Area” relates to the “Beam Cross-Sectional Area” on the “Focal Surface”.

As referred to herein, the term “Focal Surface” relates to the surface which contains the smallest of all “Beam Cross-Sectional Areas” of a focusing transducer.

As referred to herein, the term “Intensity” relates to the ultrasonic power transmitted in the direction of acoustic wave propagation, per unit area normal to this direction, at the point considered.

As referred to herein, the term “Intensity, instantaneous (I)” relates to the instantaneous ultrasonic power transmitted in the direction of the acoustic wave propagation, per unit area normal to this direction, at the point considered. It is given in the far field by:

I=P²/(ρ*c),

Wherein P is instantaneous acoustic pressure;
ρ is the density of the medium;
c is the speed of sound in the medium.

### (Unit: W/cm²)

As referred to herein, the term “Intensity, pulse-average (IPA)”, measured in units of W/cm², relates to the ratio of the Pulse Intensity Integral (energy fluence per pulse) to the “Pulse Duration”.

As referred to herein, the term “Intensity, spatial average, temporal average (ISATA)”, measured in units of W/cm², relates to the “temporal-averaged intensity” averaged over the beam cross-sectional area.

As referred to herein, the term “Intensity, spatial-peak, pulse average (ISPPA)”, measured in units of W/cm², relates to the value of the Intensity Pulse Average, IPA, at the point in the acoustic field where the IPA is a maximum or is a local maximum within a specified region.

As referred to herein, the term “Intensity, spatial-peak, temporal-average (ISPTA)”, measured in units of W/cm², relates to the value of the “temporal-average intensity” at the point in the acoustic field where the “temporal-averaged intensity” is a maximum, or is a local maximum within a specified region.

As referred to herein, the term “Intensity, temporal-average (ITA)” relates to the time average of intensity at a point in space. The average is taken over one or more “Pulse Repetition Periods”.

As referred to herein, the term “Peak-rarefactional acoustic pressure (Pr)” relates to the Maximum of the modulus of the negative instantaneous acoustic pressure in an acoustic field.

As referred to herein, the term, “Pulse Duration (PD)”, measured in units of time (seconds), relates to 1.25 times the interval between the time when the “Pulse Intensity Integral” at a point reaches 10 percent and 90 percent of its final value.

As referred to herein, the term “Pulse Intensity Integral (PII)”, measured in units of W/cm², relates to the time integral of instantaneous intensity for any specific point and pulse, integrated over the time in which the envelope of acoustic pressure or hydrophone signal for the specific pulse is non-zero. It is equal to the energy fluence per pulse..

As referred to herein, the term “Pulse Repetition Period (PRT)” for a pulsed waveform, measured in units of time (seconds), relates to the time interval between two successive pulses.

As referred to herein, the term “HIFU” relates to High Intensity Focused Ultrasound—the use of high intensity focused ultrasound energy in ultrasound treatment (therapy). Ultrasound treatment may induce a vast range of biological effects at different exposure levels. At low levels, essentially reversible cellular effects can be produced, whereas at higher intensities, instantaneous cell death may occur. Accordingly, ultrasound therapies may be broadly divided into two groups: “high” power and “low” power therapies. At the one end of the spectrum, high power therapies include, for example, high intensity focused ultrasound (HIFU) and/or lithotripsy, while at the other end, low power therapies comprise, for example, sonophoresis, sonoporation, gene therapy and/or bone healing. According to some embodiments, the term HIFU may further encompass MIFU and/or LIFU.

As referred to herein, the term “MIFU” relates to Mid Intensity Focused Ultrasound—the use of medium intensity focused ultrasound energy in ultrasound treatment.

As referred to herein, the term “LIFU” relates to Low Intensity Focused Ultrasound—the use of low intensity focused ultrasound energy in ultrasound treatment.

As referred to herein, the terms “transducing elements”, “transducing segments” and “transducing zones” may be used interchangeably. The terms relate to different regions/zones on a unitary transducer acting as individual transducers.

As referred to herein, by the terms “exciting electrode” and “apply exciting voltage to a segmented electrode” it is meant that there always exists a second (“ground”) electrode to which the same voltage but with the opposite sign is applied.

As referred to herein, the term “conductive layer” may include uniform area(s), non-uniform area(s), continuous area(s), non-continuous area(s), or any combination thereof. The term “conductive layer” is usually not limited to a layer which is necessarily conductive along its entire area; in some embodiments, a conductive layer may be a deposit of a conductive material that may be segmented earlier or later in the process, so that it is not necessarily conductive throughout.

As referred to herein, the terms “segmented electrode”, “segmented conductive layer” or “segmented layer” are referred to a plurality of electrically isolated conductive electrode elements disposed on at least one of two opposite surfaces of a unitary piece of piezoelectric material.

As referred to herein, the terms “electrode” may sometimes, when described so explicitly or implicitly, refer to a segmented layer of conductive material including multiple “electrode elements”, electrically separate from one another. For example, such an electrode may be referred to as a “segmented electrode”.

In common with diagnostic ultrasound, therapeutic ultrasound exposures can be described in terms of either the acoustic pressure or the intensity. The description of intensity for pulsed ultrasound may lead to some ambiguity. The acoustic pressure in the acoustic field is by itself spatially variant, and the pulsed shape of the signal induces additional temporal variations. It is possible to calculate intensities based on the maximum pressure measured in the field or based on a pressure averaged over a specified area. When describing the energy delivery, it is also necessary to distinguish whether the intensity is averaged only when the pulse is “on” (the pulse average) or over a longer time, which includes “on” and “off” times (temporal average). A number of different parameters related to intensity may be used. The most usual ones, defined in a number of standards (such as listed by: NEMA UD 2-1992, “NEMA Acoustic Output Measurement Standard for Diagnostic Ultrasound Equipment”, 1992, incorporated herein by reference in its entirety) are ISPTA, ISPPA and ISATA. When cavitation is the predominant mechanism, peak negative pressure is usually considered the parameter of most relevance. Table 1 hereinbelow provides a classification of ultrasound field characteristics for different applications based on values of ISPTA, frequency and pressure. The data in Table 1 is based on data from Shaw, et al, “Requirements for measurement Standards in High Intensity Focused Ultrasound (HIFU) Fields”, NPL Report DQL AC 015, National Physical Laboratory, Middlesex, UK, February 2006 and V. F. Hamphrey, “Ultrasound and Matter—Physical Interactions,” Progress in Biophysics and Molecular Biology, 93, 195-211, 2007, both incorporated herein by reference, in their entirety.

TABLE 1

| | Frequency | | |
|---|---|---|---|
| | range, | Pressure (Pᵣ), | Intensity ISPTA, |
| Modality | MHz | MPa | W/cm² |
| Diagnostic B-mode | 1-15 | 0.45-5.5 | 0.0003-0.99 |
| Diagnostic CW Doppler | 1-10 | 0.65-5.3 | 0.17-9.1 |
| Bone growth stimulation | 1.0-1.5 | 0.05 | 0.03 |
| Physiotherapy | 0.75-3.4 | 0.5 | <3 |
| Drug delivery | Up to 2.0 | 0.2-8.0 | Various |
| | | | intensities |
| HIFU thermal | 0.8-2.0 | 10 | 400-10000 |
| HIFU histotripsy | 0.7-1.1 | 22 | 200-700 |
| Haemostasis | 1-10 | 7 | Up to 5000 |
| Lithotripsy | 0.5 | 10-15 | Very low, |
| | | | <10-4 |

In general, there are a few ways by which ultrasonic waves may influence a tissue with which they interact: thermal (heating) effects, and/or mechanical effects (such as, for example, shearing forces, cavitation, and the like), as further detailed hereinbelow.

Several therapeutic ultrasonic applications use heating to achieve a required effect. In the case of “low power” ultrasound, raising the temperatures above normothermic levels by a few degrees may have a number of beneficial effects, such as, for example, increasing the blood supply to the affected area. In case of “high power” ultrasound applications, tissue temperature is raised very rapidly (typically in less than 3 seconds) to temperatures in excess of 56° C. This may usually cause instantaneous cell death. For example, hyperthermia treatments rely on cells being held at temperatures of 43-50° C. for times up to an hour, which may lead to the inability of cells to divide. The magnitude of the temperature rise depends on the ultrasound intensity, the acoustic absorption coefficient of exposed tissue, tissue perfusion and time for which the sound is “on”. The temperature increase due to ultrasound absorption can be calculated by using Pennes bio-heat equation (H. H. Pennes, “Analysis of issue and arterial blood temperatures in the resting human forearm, J. Appl. Physiol. 1, 93-122, 1948, incorporated herein by reference, in its entirety):

$$\frac{d T}{d t} = k {∇}^{2} T - \frac{( T - {T}_{0} )}{τ} + \frac{{q}_{v}}{{ρ}_{0} {C}_{P}}$$

wherein, k is the thermal diffusivity, r is the time constant for the perfusion, T₀ is the initial (ambient) temperature, qᵥ is the heat source distribution and Cp is the specific heat capacity of the medium at constant pressure. The first term on the right-hand side of Pennes bio-heat equation accounts for diffusion using the gradient of temperature while the second term accounts for perfusion using the diffusion time constant.

In general, the heat source term qv is very complex as it depends on the nature of the field produced by the transmitting transducer, which may be, for example, focusing. There exist a number of approaches for calculating qᵥ. One of them, which is valid even for strongly focusing transducers and high amplitude values, is described by, for example, Goland V., Eshel Y., Kushkuley L. “Strongly Curved Short Focus Annular Array For Therapeutic Applications,” in Proceedings of the 2006 IEEE International Ultrasonics Symposium., 2345-2348, Vancouver, 2006, the content of which is incorporated herein by reference, in its entirety.

Several therapeutic ultrasonic applications use mechanical effects to achieve desired results. The most prominent of the mechanical effects are shearing force (stress) and cavitation. The term cavitation generally refers to a range of complex phenomena that involve the creation, oscillation, growth and collapse of bubbles within a medium. The cavitation behavior depends on the frequency, pressure, amplitude, bubble radius and environment. For example, lithotripsy therapeutic procedure uses focused shock waves at very high acoustic pressure for destroying stones in kidneys. Since in this application the repetition frequency of pulses is very low (at about 1 Hz), there is no noticeable heating during the treatment, and the produced effect can be considered as solely mechanical. Another example of the mechanical effect related to cavitation is histotripsy procedure, which is defined as mechanical fractionation of soft tissue by applying high-amplitude acoustic pulses with low temporal-average intensities. Its mechanism is a non-thermal initiation and maintenance of dynamically changing “bubble clouds”—a special form of cavitation, which is used for precisely destroying tissue such as in cardiac ablation.

When the signal amplitude is under the cavitation threshold but still high enough, then shear stresses may be responsible for biological effects. It has been previously shown (For example, by Burov et al., nonlinear Ultrasound: Breakdown of Microscopic Biological Structures and Nonthermal Impact on a Malignant Tumor”, Doclady Biochemistry and Biophysics, 383, 101-104, 2002, the content of which is incorporated herein by reference in its entirety) that exposure of cells to high power ultrasonic radiation, under the conditions excluding thermal and cavitation-induced degradation, was accompanied by structural modification of macromolecules, membranes, nuclei and intracellular submicroscopic complexes. Some of the mechanisms that were suggested to explain these phenomena are: large shear stresses generated in the thin acoustic interface near solid boundaries, forces of friction between large-mass macromolecules and surrounding oscillating liquid, acoustic microscopic flows, or any combination thereof.

A parameter that allows estimating the likelihood of cavitation is called Mechanical Index (MI) and is defined as:

$$M I = \frac{{P}_{r}}{\sqrt{f}}$$

wherein Pr is the peak rarefactional pressure of the acoustic signal in MPa and f is the frequency of the signal in MHz. The American Institute of Ultrasound in medicine (AIUM), National Electrical Manufacturers Association (NEMA) and FDA adopted the Mechanical Index as a real time output display to estimate the potential for cavitation during diagnostic ultrasound scanning (see “Standard for Real-Time Display of Thermal and Mechanical Acoustic Output Indices on Diagnostic Ultrasound Equipment”, 2nd ed., AIUM, Rockville, 1998, incorporated herein by reference). The assumption is that if one does not reach the threshold MI=0.7, then the probability of cavitation is negligible. The maximum value of MI that is allowed for diagnostic machines seeking approval in the USA is 1.9. For example, it has been previously shown experimentally, that MI values, which correspond to a cavitation threshold at a frequency of, for example, 0.2 MHz, have values from 3.4 to 7.8, depending on tissue type and characteristics.

Therefore, it may be understood that by choosing the appropriate set of signal parameters one can expose tissue in “thermal” and/or “mechanical” mode, causing various or completely different effects. If, for example, the signal amplitude will be under the cavitation threshold, but the energy is delivered in continuous mode (CW), or at high DC values, then the effect may be mostly thermal. At high ISPTA values, coagulation and necrosis of tissues may be caused. Changing DC values, it is possible to vary temperature limits and its rise rate in a wide range. By contrast, by choosing very high signal amplitudes (over the cavitation threshold) and very low DC, it is possible to produce mechanical effects causing negligible heating. At high ISPPA and low ISPTA values, one can achieve complete tissue emulsification without heating. Tissue debris size in this case may be as little as 2 μm. Hence, selection/use of appropriate parameters may permit selective formation of cavitation in target tissue but not in neighboring tissues.

Ultrasonic energy can be non-invasively delivered to the tissue in either a non-focused or focused manner. In the first case, tissue is exposed to approximately the same extent, beginning from the skin and down to a certain depth. Due to ultrasound attenuation in the tissue, the signal energy will decrease with distance so that the maximum intensity will be on the skin. Beam divergence for non-focused ultrasound is very low; it begins to increase only from distances Z>d²f/4c from the radiator surface, wherein d is a characteristic dimension of the radiator (such as a diameter). For example, for a radiator having a diameter of 30 mm and working at 1.0 MHz, this distance will be of about 150 mm. This means that the ultrasound energy target non-selectively all types of tissue (skin, subcutaneous fat, muscles, and so forth) within the cylinder with a diameter of 30 mm and height of at least 150 mm. The maximal energy that could be delivered at a certain depth (where the effect is sought for) is limited by the levels, which are considered safe for surrounding tissues (including skin). Focused ultrasound allows overcoming these problems by concentrating most of the energy in the focal area, where the intensity is significantly higher than in the surrounding tissue.

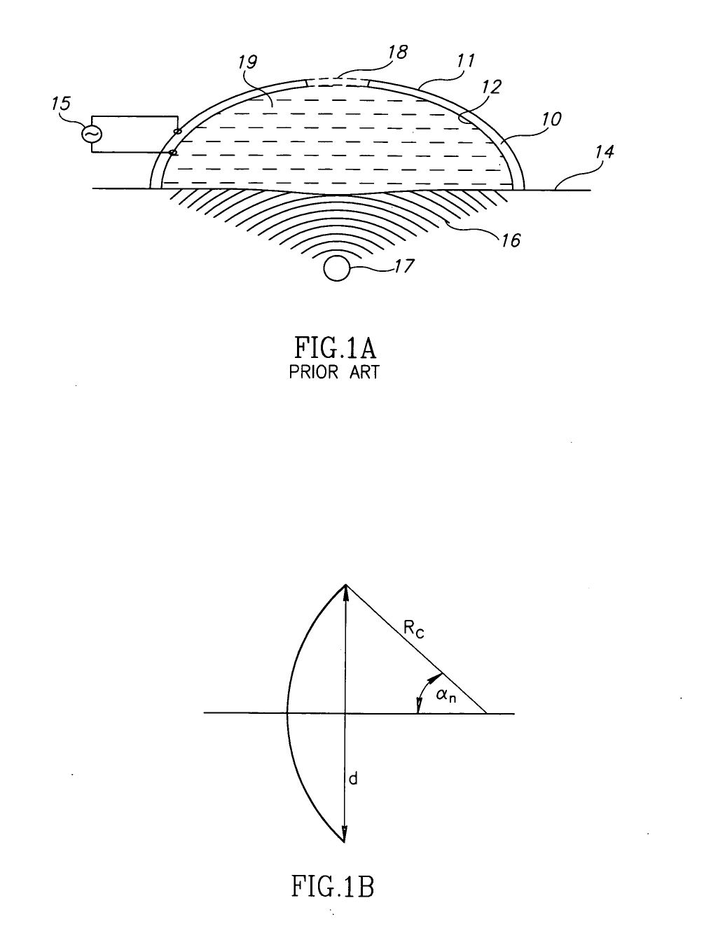

Reference is now made to FIG. 1A, which illustrates schematically a cross sectional view of a prior art ultrasonic hemi-spherically shaped focusing piezoelectric transducer 10, typically being used to provide high intensity focused ultrasound (HIFU) to lyse adipose tissue in a tissue region of a patient's body below the patient's skin 14. The transducer 10 may be produced using any of various methods and devices known in the art, and is formed having electrodes 11, 12, in the form of thin conducting coatings on its surfaces. The transducer is driven by means of a high frequency power source 15, which applies a voltage between the electrodes 11, 12, of the transducer, thus exciting resonant vibration modes of the transducer, and generating high intensity ultrasound waves for killing, damaging or destroying adipose tissue. The transducer is optionally filled with a suitable coupling material 19 for acoustically coupling the transducer to the patient's skin 14. A commonly used material is a gel. Because of the concave shape of the transducer, the ultrasound waves are focused 16 towards a focal region 17, which is generally in the form of an ellipsoid, having its major axis along the wave propagation direction. The size of this focused region is dependent on a number of factors, mainly the curvature of the transducer and the frequency of ultrasound emitted, varying for a transducer in the order of 70 mm diameter, from an ovoid of approximately 7 mm×5 mm for a frequency of 200 kHz, to approximately 3 mm×1.5 mm for 1 MHz ultrasound. A hole 18 is provided at the apex of the transducer, for placing an imaging transducer for monitoring acoustic contact and/or treatment efficiency during use of the transducer. It is to be understood however, that this monitoring can also be accomplished by using any of the electrodes of the array, such that the central hole monitor is only one method of performing the monitoring, and where optionally illustrated in any of the drawings, is not meant to limit the transducer shape shown.

The frequency of the emitted ultrasound, for a transducer of given shape, material and diameter, is mainly dependent on the thickness of the shell. For instance, for an 84 mm diameter cap-shaped transducer similar to that shown in FIG. 1A, for a thickness of 8.4 mm, a transducer using a ceramic of the type APC841, supplied by Americam Piezo Ceramics, Inc., PA, USA, will emit at a frequency on the order of 200 kHz, while for a thickness of 1.7 mm, the transducer will be excited at a frequency on the order of 1 MHz.

Furthermore, considering the spherical segmented transducer schematically illustrated in FIG. 1B, having an aperture diameter d, radius of curvature Rc and working frequency f, the expression for pressure gain K_{P}, which is a ratio of pressure P_{F} in the focus to pressure P_{S} on the radiator surface may be provided by the formula:

$${K}_{P} = \frac{{P}_{F}}{{P}_{S}} = \frac{2 π \cdot f {R}_{C}}{c} ( 1 - cos {α}_{n} )$$

wherein αₙ is a half-aperture angle. Analysis of the equation demonstrates that it is possible to increase the gain by increasing either f or αₘ or both. For example, a radiator with d=100 mm and Rc=100 mm will have Kp=11 at frequency 0.2 MHz and Kp=55 at 1.0 MHz.

As mentioned hereinabove, interaction of the focused ultrasound waves with the tissue on which they are focused is dependent on a number of factors: thermal effects, which usually result in coagulation of the tissue, and are non-selective, the acoustic energy affecting whatever tissue it encounters at a power density at which the effects take place; rupture or mechanical effects, which tear the cell walls, thus damaging the cell structure itself. This may not destroy the cell immediately, but may damage it sufficiently that it dies within a period following the treatment. This may be hours or days, depending on the extent and type of damage inflicted. This phenomenon is generally highly selective with regard to the type of tissue on which the ultrasound impinges, but it requires a high level of energy on target to be effective. Such mechanical effects may include streaming, shear or tensional forces, and cavitation effects, in which small air bubbles are formed within the tissue.

The treatment time per patient, using a current, state-of-the-art, roving focusing ultrasonic head, such as the one illustrated in FIG. 1A, treating successive regions at a time, is typically 90 minutes, and may involve almost 1,000 treatment nodes to cover an adult abdomen, each spot taking approximately 6 seconds. Generally, only about half of this 6 second period may be spent in actual treatment, the rest of the time being used for moving and positioning the treatment head. For reasons of commercial efficacy, and for reasons of patient acceptance, it would be highly desirable to significantly decrease this time. Prior art methods of achieving this generally rely on increasing the total energy of ultrasound applied to the tissue, thus reducing the time needed to achieve the desired effect. There are a number of ways of doing this, such as, for example: increasing the exciting voltage applied to the transducer, which, increases the intensity of the ultrasound waves emitted; increasing the duty cycle of the pulses in the pulse train applied, to provide higher averaged power; and the like.

These methods are known in the art. However, it is not always possible or desirable to increase the operating frequency because sound attenuation increases with higher frequencies, and this may lead to higher heating and decreasing of a penetration depth of the ultrasound. In addition, since focal area dimensions are of the order of magnitude of the wavelength, higher frequencies produce smaller focal areas, thus limiting treatment abilities or increasing treatment time. Increasing the half-aperture angle an (FIG. 1B) requires enlargement of the transducer, making it more heavy and expensive, and less suitable for work. Moreover, some of the methods described above generally result in increased cavitation, or increased thermal effects, both of which are non-selective and, hence, may be dangerous to organs and/or tissue which are in close proximity to the treatment region. Furthermore, both these effects ultimately involve increased pain to the patient, which may make the treatment unacceptable. One prior art system utilizing a planar applicator, which results in a sheet of tissue being treated, in order to achieve faster results, operates intentionally in the thermal damage range of power, such that the patient's skin has to be continuously locally anesthetized for the treatment to be bearable.

Further methods of increasing the efficacy of the treatment may be obtained by using the phenomenon known as Time Reversal, as further expounded in applicants' U.S. patent application Ser. No. 12/003,811, entitled “Time Reversal Ultrasound Focusing”.

There are potential advantages to the variously available HIFU procedures, in the use of a number of separate transducers, each of which can be excited separately, rather than using a single transducer working in a single mode of operation. There exist a number of methods of constructing such multiple transducer ultrasound heads. One of the simplest is to simply construct the spherical emitter out of a number of assembled segments of separate transducers. Additionally, in U.S. Pat. No. 7,273,459 for “Vortex Transducer” to C. S. Desilets et al., there is described a method by which a multiple transducer head is produced by embedding a large number of separate transducer elements, each diced from a single transducer, in a matrix of epoxy.

Such methods of construction may generally be costly, time consuming, may possibly have a limited yield, and, because of the loosening effect of high intensity ultrasound on the glue or epoxy, may have a limited lifetime. Furthermore, the adhesive may also absorb part of the ultrasonic energy, thus limiting power efficiency.

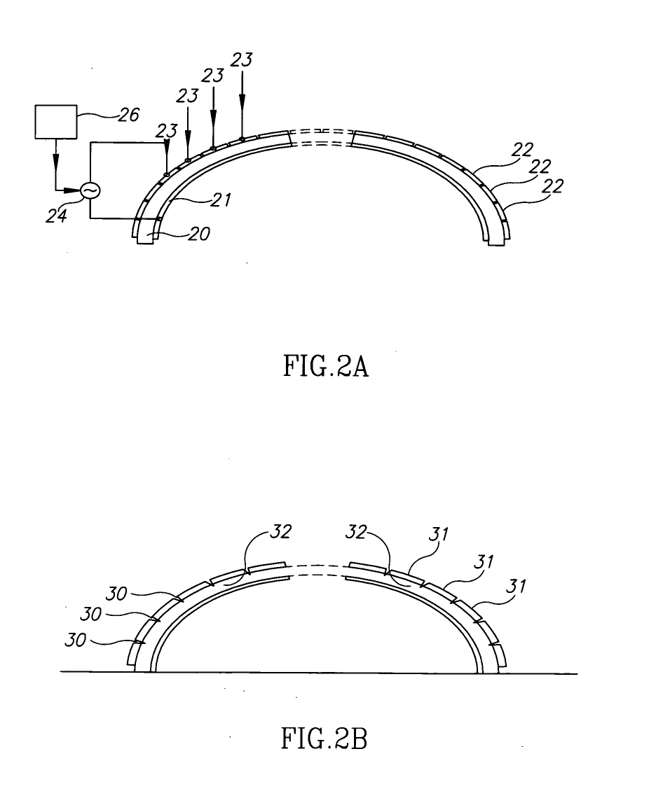

Reference is therefore made to FIG. 2A which illustrates schematically, a multiple transducer head, constructed according to an embodiment of the present disclosure, which utilizes a single ceramic element, virtually divided into separately emitting sub-transducers by means of dividing one of the exciting electrodes into electrically-separate electrode elements. In FIG. 2A, there is shown a cross sectional view of a spherical ultrasound transducer 20, comprising a piezoelectric ceramic material which emits the ultrasound waves when excited. One surface of the transducer 20 may have a continuous conducting electrode, 21, while the electrode on the opposite side may comprise a number of electrically separate electrode elements 22, each of which may be excited by application of the appropriate predetermined high frequency voltage by means of connecting leads 23. In FIG. 2A, for clarity, the exciting source 24 is shown connected to only one of those electrode elements, though it is to be understood that each of the electrode elementsshould be so connected, either each independently of the others to its own high frequency voltage source, or alternatively, together with several groups of electrode elements, each group being connected to a separate source, or alternatively, together with all of the other electrode elements, all being connected to a single source. The voltage source or sources may be activated by means of a controller 26, which may be programmed to emit pulses for a predetermined length of time and at a predetermined rate and duty cycle commensurate with the treatment being performed. For convenience, it is the outer electrode of the arrangement of FIG. 2A which is shown segmented 22, this enabling simpler application of the exciting power, although it is to be understood that the disclosure will operate equally well with the inner electrode 21 segmented. It is even possible for both of the electrodes to be segmented, inner and outer segments generally being arranged opposite each other, but this arrangement may unduly complicate the electrical connection requirements.

The production of the separate electrode elements can be achieved by any of the methods known in the art. One such method is the coating of a continuous conductive layer, followed by mechanical scribing of the layer, whether the scribing is such that it penetrates into the ceramic surface itself, as shown in scribe marks 30 which penetrate into a ceramic surface 32, or whether the scribing only cuts the electrode into its separate elements, as shown in elements 31, both as shown schematically in the embodiment of FIG. 2B. The scribing process can be performed on one surface only, or on both surfaces. This process can be a mechanical scribing or cutting process, or an ablating process, such as can be efficiently and rapidly performed using a CNC controlled laser scribing machine.

Alternatively, the electrode elements can be applied in an already segmented form by any of the methods known in the art, such as by silk screen printing, by spray or brush or roller painting or by vapor deposition or sputtering through a mask. By this means, the electrode elements can be applied in a particularly cost effective manner, since all of the separate electrodes are formed in a single procedure. Furthermore, the electrode elements can be readily applied on a base transducer having any shape or profile, whether spherical, flat, cylindrical, or the like. All that is required is a suitably shaped mask to fit to the contour of the transducer surface on which the segmented electrodes are to be coated. Additionally, because of the blanket method of generating the electrode elements in a single process, there is no limit to the number of electrode elements which can be produced, in contrast to prior art methods where each electrode element, or segment, requires individual handling. It therefore becomes practical to make transducer heads with very large numbers of electrode elements, which increases the flexibility and accuracy with which the various applications of the present disclosure can be performed.

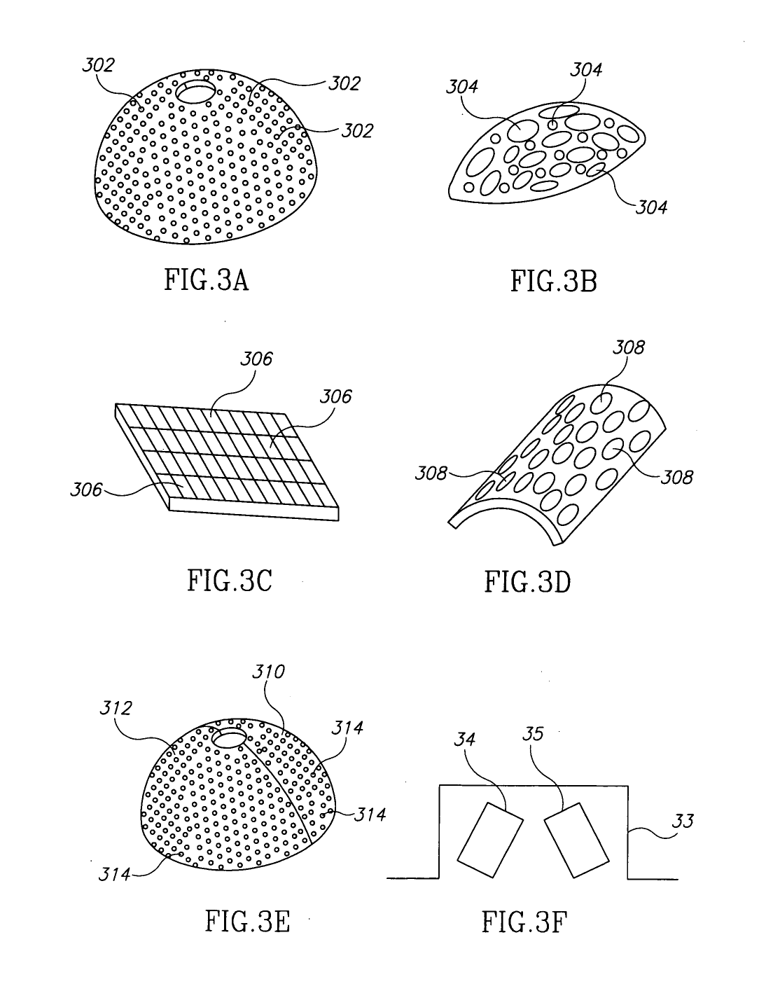

Reference is now made to FIGS. 3A to 3F, which illustrate schematic views of various differently shaped transducers, each comprising a single unitary piece of ceramic as the base, and having a plurality of electrode elements (or, in short, “elements”) on one of its surfaces. FIG. 3A shows a plurality of circular elements, such as elements 302; FIG. 3B is a similar embodiment but showing how elements of different size, such as elements 304, can also be used; FIG. 3C shows a flat transducer having elements such as elements 306; and FIG. 3D shows a cylindrically shaped transducer having elements such as elements 308. The cylindrical embodiment of FIG. 3D provides a line of focused energy instead of a spot, and this may be useful for treatments performed on the arm or leg of a subject. It is to be understood that the arrangement of elements can be of shapes other than circular, can be randomly or regularly positioned, or can be loose-packed or close-packed or tiled, without departing from the present disclosure. Thus, in the embodiment of FIG. 3C, the electrode elements are shown in the form of a tiled rectangular array, which could be produced by simply scribing the rectangular lattice on the coated electrode, or by coating through a rectangular lattice. Such tiled arrangements utilize essentially all of the area of the transducer surface. Other tiled arrangements could also be used, such as squares, triangles (alternately inverted), hexagons and others. In addition, the use of various patterns and shapes such as circles, ovals, octagons, and the like, which do not form tiled structures, may also be used and may result in at least partial utilization of the transducer surface area.

Furthermore, although the transducer head is most simply constructed using a single piece of piezoelectric material for the base element, as shown in the embodiments of FIGS. 3A to 3D, there may be applications or head shapes or sizes which make it preferable for the base element to be constructed of more than one piece of piezoelectric material, such as is shown in FIG. 3E, where the base piezoelectric element is made of two pieces of piezoelectric material 310, 312, each of which is separately divided into sub-transducers by means of the electrode element arrangement of the present disclosure, shown at elements such as elements 314. Likewise, the head could comprise an array of separate transducer elements, each of the separate transducer elements being itself made up of a single unitary piece of transducer material, operated as a multi-transducer by virtue of the multiple electrode elements coated on it.

Reference is also made to FIG. 3F, which illustrates a head 33, made of two completely separated transducers 34, 35, which are operated in co-ordination to produce the desired focusing effects.

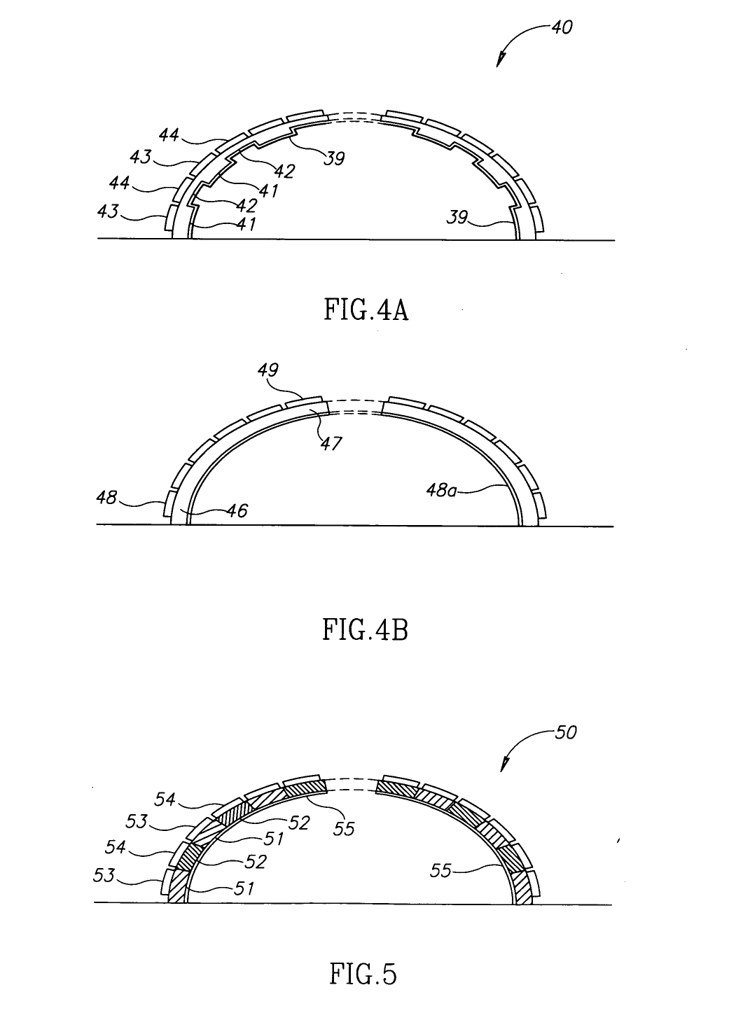

Some applications of HIFU treatments require the use of ultrasound of different frequencies, or of combinations of frequencies, as outlined in applicants' U.S. Provisional Patent Application No. 61/064,582, entitled “Patterend Ultrasonic Transducers”. There are a number of ways in which such an output can be generated from a transducer head constructed according to various embodiments of the present disclosure. Reference is now made to FIG. 4A, which illustrates schematically an embodiment of a transducer head 40, according to the present disclosure, constructed to operate at multiple frequencies. The base piezoelectric transducer material is of similar shape to that of the embodiment shown in FIG. 1A except that it is constructed with regions having different thicknesses. Thus in region 41, the material is thicker than in region 42. Using the exemplary data given for the embodiment of FIG. 1A, if the thinner regions 42 are made to be in the order of 1.7 mm thick, they will emit at approximately 1 MHz, while for an 8.4 mm thickness of the thicker regions 41, the frequency will be in the order of 200 kHz. The positions of the electrode elements can be arranged such that they generally overlap the positions of the different thickness regions, each of the thickness regions 41, 42, having their own individual exciting electrode elements 43, 44, such that it is possible to excite each frequency according to the electrode elements which are activated. The inner surface may have one or more electrodes and/or electrode elements, such as, for example, electrode 39. Thus, when an electrode elements 43 is activated, a 200 kHz beam is emitted from the section of piezoelectric material 41 below it, while activation of electrode elements 44 results in a 1 MHz beam. By activating both sets of electrodes together, or by activating at least some of each of the electrodes together, it also becomes possible to treat the target area with two frequencies simultaneously, which may be advantageous. Additionally, it may be possible to excite heterodyne frequencies arising from beating of the two frequencies, if the ultrasound emitted from the two sets of electrodes impinge together on the target zone. The embodiment of FIG. 4A shows only two different thickness regions, although it is to be understood that a larger number of different thicknesses can also be implemented, each thickness region vibrating at its own characteristic frequency.

Although the embodiment of FIG. 4A shows sharp transition steps between the different thicknesses, it is to be understood that the transitions can also be gradual. Such an embodiment is shown in FIG. 4B where the thickness of the transducer material is gradually changed across the width of the transducer, being in the example of FIG. 4B, thicker 47 in the center of the transducer, and thinner 46 at the extremities. A range of frequencies can then be emitted by such a transducer. Thus, when electrode elements such as 49 are excited at the appropriate frequency, the emitted vibrational frequency is lower than, for instance, electrode elements such as 48. The inner surface may have one or more electrodes and/or electrode elements, such as, for example, electrode 48a.

An alternative method of generating different frequencies is shown in FIG. 5, which shows schematically a single unitary element transducer 50 having regions of different material characteristics or constitution, such that they vibrate at different frequencies. The different regions can be of either different stoichiometric composition, or of different doping levels, or of different densities, all as determined by the mixing and firing methods used for producing the ceramic, if the piezoelectric material is a ceramic. In the example shown in FIG. 5, two different types of region are shown, one type being designated by the cross hatching 51, and the other by the longitudinal shading 52. Each region has its own characteristic electrode elements, 53, 54, located to excite just that region in juxtaposition to the electrode, such that application of the activating voltage to one or other of the electrode elements 53, 54, can result in different frequency ultrasonic beams being emitted. The inner surface may have one or more electrode elements, such as, for example electrode 55. The embodiment of FIG. 5 shows only two types of transducer regions, although it is to be understood that a larger number of different types of regions can also be implemented, each type vibrating at its own characteristic frequency.

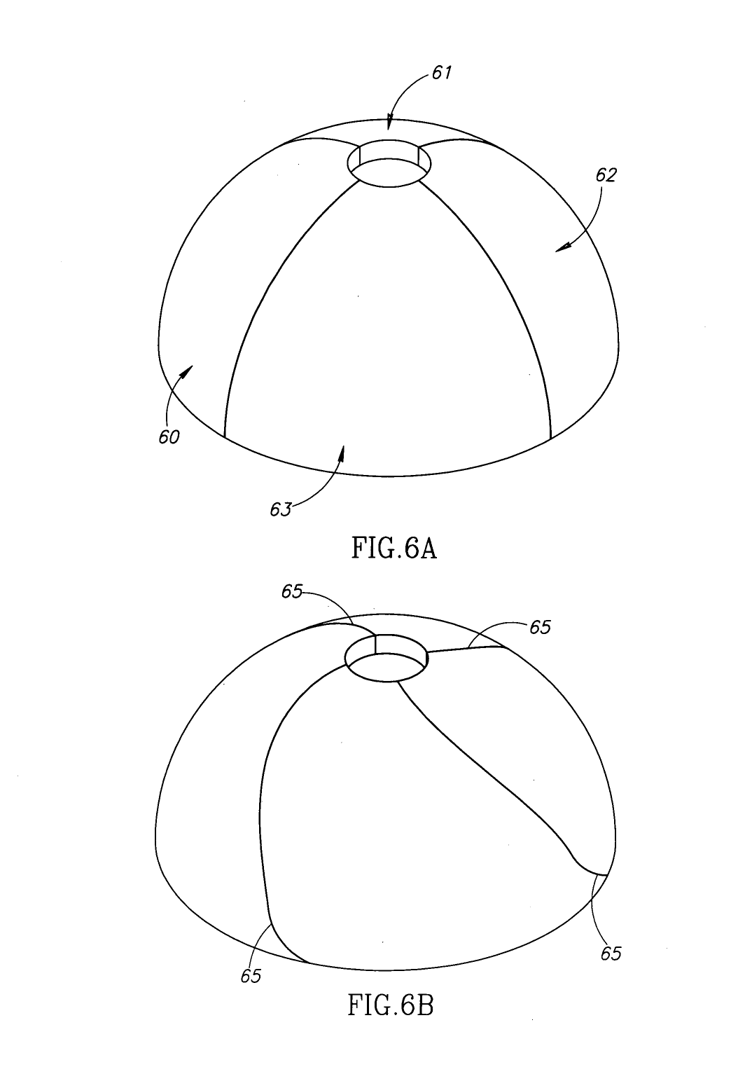

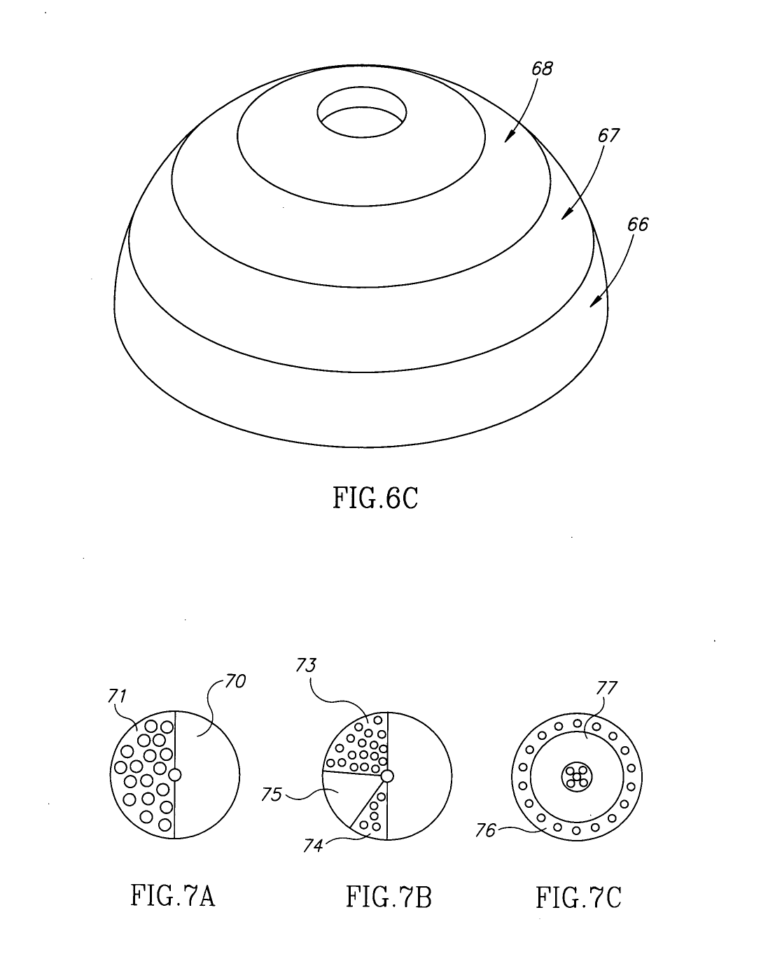

In the above described transducer heads, the electrodes have been comparatively small, such that the transducer is made up of a large number of separate segmented transducers by virtue of the electrode elements. According to different embodiments, this number can run even up to over one hundred transducer segments, such a division being difficult to execute without the segmented electrode technology of the present disclosure. Cutting and sticking together such a large number of small elements is a difficult task to perform reliably and cost-effectively. However, it is to be understood that the present disclosure also provides advantages for embodiments where there are only a small number of segments in the transducer, starting with only two segments. As previously stated, the degrading effect of high power ultrasound on any adhesive joint may affect such assembled multiple segment transducers. Therefore, there are advantages even in a two- segment transducer using a single ceramic base transducer, and a segmented electrode constructed and operative according to the methods of the present disclosure. Reference is now made to FIGS. 6A to 6C, which illustrate schematically some additional possible arrangements of segmented electrode transducer elements with such a small number of segments. FIG. 6A illustrates in plain schematic view, a four-segment transducer constructed of a single piece of piezoelectric material with four separate electrodes 60-63, coated thereon, each electrode being separately excitable by means of its own applied voltage. The four segments could have different thicknesses, or different properties, as described in the embodiments of FIGS. 4 and 5, such that each segment vibrates at a different frequency. FIG. 6B shows a transducer with a quadruple segmented electrode pattern, the inter-electrode elements boundary lines having a curved “S” shape 65. Use of such an embodiment may possibly have some specific effects on the tissue, and use of the segmented electrode technique of the present disclosure considerably simplifies the task of manufacture of such a transducer. FIG. 6C shows another embodiment of a transducer with concentric electrode regions 66, 67, 68, applied to a single ceramic transducer element. Such an embodiment is useful for generating different phased emissions. It is to be understood that FIGS. 6A to 6C are only some of the possible shapes which can be constructed using the segmented electrodes of the present disclosure, and that this aspect of the disclosure is not meant to be limited to what is shown in exemplary embodiments of FIGS. 6A to 6C.

Alternatively, some of the segments could themselves have a segmented pattern of electrode elements, such that the transducer head acts as a combination of large segment transducers, and an array of small segmented transducers.

Reference is now made to FIGS. 7A to 7C, which illustrate schematically some additional possible arrangements of arrays of separate transducer elements, any of which may itself be operative as a multi-segmented transducer by virtue of an assembly of electrode elements on its surface, such that the transducer head acts as a combination of large segment transducers, and an array of small segmented transducers. The embodiment of FIG. 3F above shows one example of a transducer head made up of two separate unitary multi-segmented transducers. The embodiments shown in FIGS. 7A and 7B illustrate how the arrangement of these arrays can be symmetric, as shown in FIG. 3E, or non-symmetric, if such a non-symmetric arrangement is desired for the application at hand. FIG. 7A shows a spherical transducer head, having 2 separate sectors, one of which is a single piece, single segment transducer 70, and another sector 71 having electrode elements over its surface. FIG. 7B shows an exemplary embodiment in plain view, in which there is a single piece array 73 covering a quarter of the transducer head, another multi-electrode element, single piece array 74 covering one eighth of the transducer head, and a further single piece, single electrode transducer 75 covering another eighth of the transducer head. FIG. 7C shows a cap with annular sections, similar to that shown in FIG. 6C, in which one section 76 is made up of a number of segmented annular sections, electrode transducers, some of which are single piece, multi-electrode element transducers with a large number of segments thereon, and other sections 77 being single piece, single transducers. Other combinations and arrangements are also possible, as will be evident to one of skill in the art.

The application flexibility afforded by the above-described unitary, piezoelectric, multiple electrode element transducer heads enables a number of novel ultrasound treatment applications to be performed, some of which have been mentioned hereinabove in connection with the construction details of the transducer heads. These novel uses and applications are broadly based on the use of multiple transducer arrays in an analogous manner to the phased arrays used for instance, in radar technology. With such an array of transducers, the position and phase of every ultrasound emitting point is known, and by correct summation of these multiple emissions, it is possible to both direct and to shape the emitted beam and its focal shape in the target area. A controller function is required to ensure that each segment used to build the beam vibrates at the correct time, with the correct amplitude, and with the correct phase, relative to the other segments taking part in the emission. The arrays can be operated either in a pure phased array manner, in which case the phase and amplitude of the various transducers contributing to the treatment are controlled in a predetermined manner, or in a scalar array manner, in which separate transducers in the array are excited either sequentially or coincidentally, but without any specific phase relation between the exciting fields, and the results combined additively.

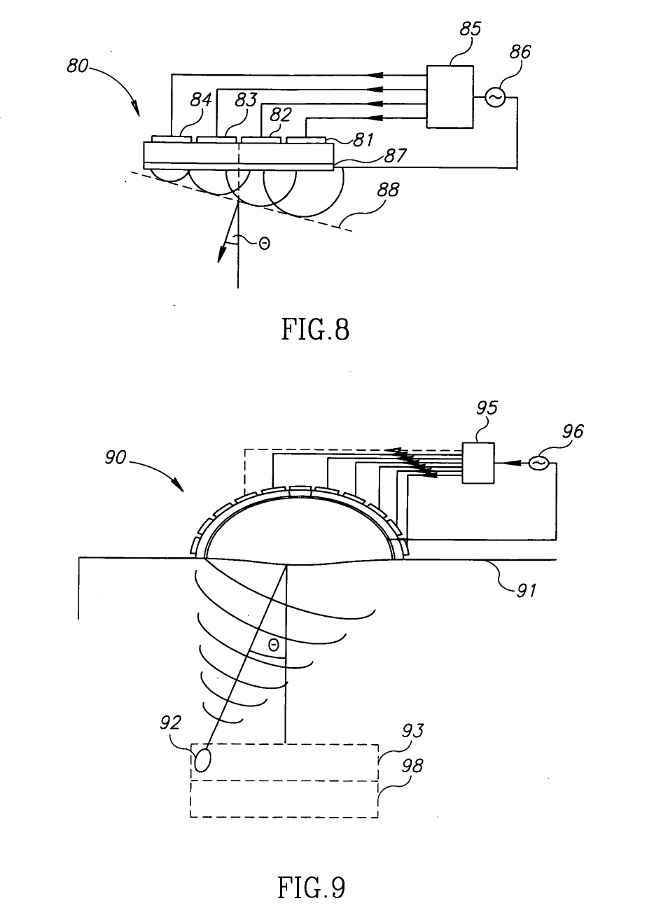

Reference is now made to FIG. 8, which illustrates the manner in which an array of transducers, excited as a phased array, can direct the emitted beam of ultrasound. FIG. 8 illustrates schematically a method of beam steering using a flat array of transducers, such as that shown in FIG. 3C. The array 80 may comprise a plurality of separate transducer element segments, each defined by its electrode element 81, 82, 83, 84, driven through a controller 85 from a high frequency exciting voltage 86 applied between the segment being addressed and the opposing electrode 87. The controller may be programmed to begin the emission of a pulse from each transducer element at a slightly delayed time from the preceding transducer element. A time “snapshot” of the propagating wavefronts from all of the transducer elements thus shows that the emission from the first element 81 has propagated further than that of the second element 82, and that of the second element 82, further than that of the third element 83, and so on. A line drawn connecting all of the wavefronts shows that the resultant wavefront of the ultrasound 88 is propagated at an angle θ to the normal to the phased array, where the angle θ is a function of the time delay (and hence phase) between the various emitting elements. Although the controller 85 is shown in the embodiment of FIG. 8 as a form of schematic switching device, directing the voltage generated in high frequency source 86 to the various electrode elements 81, 82, 83, 84, it is to be understood that this is only one possible non-limiting arrangement for exciting the electrode elements of the transducer segments, and that other electrical arrangements, such as individual controlled oscillators for each electrode element, or for groups of electrode elements, could equally well be used in the present disclosure. This is also so for the other phased array embodiments described hereinbelow.

FIG. 8 shows a simple ultrasound beam steering application, which is one of the simplest forms of time-domain, phased array beam manipulation. However, more complex patterns of control, including patterns executed by the use of frequency domain control, can also be used to perform more complex manipulation of the ultrasound beam. Such more complex operations may include the insertion of zeroes into the beam propagation characteristics, or the cancellation or amendment of side lobes, both of which can be achieved by multiplication of the emitted beam power using a predetermined window factor across the transducer array. Other effects include the variation of the size and shape of the focus region, as is known in the art of phased arrays.

Thus, by use of a phased array of transducers, a number of operational results can be achieved which are effective in improving the treatment parameters in focused ultrasound applications, and especially the important parameter of reducing the time of treatment. Firstly, the use of a phased array transducer generally enables the beam direction, the beam shape, and the beam energy profile to be more accurately determined and controlled than using other applicators. This enables accurate spatial application of the ultrasound energy. Such accurate placement of energy enables treatment to be performed without affecting closely lying organs, especially in those applications where non-selective conditions are used. Additionally, because of this increased positional accuracy, treatment can be performed closer to the skin without engendering undue pain from the nerve endings close to the skin. Another advantage of the accurate control of the ultrasound energy made possible by the use of phased array transducers is that the ultrasound energy can be applied at a predetermined intensity level needed to treat a predetermined region with a desired type of ultrasound interaction, for instance, selective mechanical effects rather then non-selective thermal effects. This closer control of energy also provides additional safety against undesired damage to tissue. Furthermore, the beam focal point can be swept across a region to be treated without motion of the transducer head. Furthermore, the focal plane of the ultrasound beam can be varied by the appropriate excitement conditions applied to the segmented transducers. Thus, instead of a treatment volume limited to the size of the ellipsoidal focus of the single transducer, such as, for example, the 5 mm×3 mm region mentioned above for a 1 MHz spherical transducer head, beam sweeping may make it possible to cover a cube of dimensions 15 mm×15 mm×15 mm or more, without moving the transducer head. This saving of the time taken in moving the head can reduce the time of a treatment significantly. Additionally, the focus region of the ultrasound beam can be tailored to achieve a treatment region having a predetermined shape and power density profile. All of these parameters can be selected to increase the effectiveness, speed and selectivity of the treatment without generating pain, and without invoking undesired and undue effects, such as, for example, thermal effects and/or damage to tissue/areas other than the target area and treatment volume.

Reference is now made to FIG. 9, which illustrates schematically the effect of the application of the beam steering technique shown in FIG. 8, to a cap-shaped segmented transducer 90, such as that shown in FIG. 2A or 2B, applied to a subject's skin 91. The time delays applied to successive segments for any deflection angle may need to be different from the linearly increasing time delays used in the embodiment of FIG. 8, because of the curved nature of the transducer head. The point of focus 92 of the ultrasound beams can be moved to different angles θ according to the time delay applied to successive electrode elements by the controller 95, driven by a high frequency exciting voltage 96.

By programming the controller 95 to vary the time delays in a continuous manner, a simple beam sweep can be obtained, enabling the coverage of a larger target area than would be obtained from the focused static ultrasound beam. This is shown in FIG. 9 by the dotted outline area 93, which can be significantly larger than the size of the static focused region. Additionally, the targeted regions can be arranged, by selective phased firing of the different transducers or groups of transducers, to lie not only side by side, but also in different planes, such that an extended volumetric region of treatinent in all three dimensions can be obtained. This depth of treated volume is illustrated in FIG. 9 by the targeted region 98.

Although a classic phased array application generally involves the interaction of a number of beams, whose mutual phases have been adjusted to produce an interference pattern which generates the desired effect on target, it is to be understood that the elements of the array of segmented transducer elements of the present disclosure can also be activated sequentially, or in a combined sequential/parallel manner, in order to achieve further possible advantages.

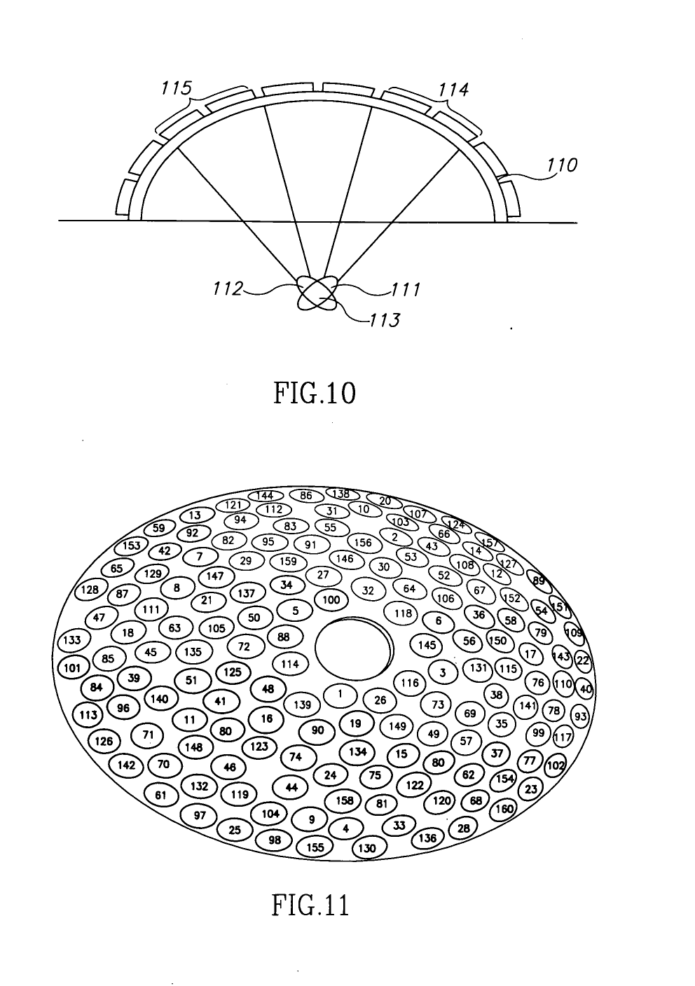

According to a further embodiment, as shown in FIG. 10, it is also possible to fire two transducer segments or groups of transducer segments together, thus enabling the attainment of an additive power level on target which would not be attainable by each transducer or group of transducers alone. The cap transducer 110 has different groups of transducers which can all be directed to fire at a common focus point within the subject's tissues. Thus, the transducers in the region of the electrode elements 114 produce a focused volume in the form of an ovoid 111 aligned at one angle, while the transducers in the region of the electrode elements 115 produce a focused volume in the form of another ovoid 112, essentially in the same position, but aligned at another angle. Where the two ovoids overlap 113, the energy density achieved is greater than that achievable by either of the two ovoids separately. Furthermore, even if the two transducers or groups of transducers cannot be fired simultaneously, it is possible to fire them sequentially, and so long as the firings are sufficiently close, the effect on the tissue may be additive. At the same time, an advantage of this additive energy system is that for locations other than the target region, the power density is below the level of damage to the tissue, such that tissue neighboring the target zone is not affected. A specific application of this aspect of the present disclosure could be used to apply two focused beams of ultrasound, each less than the level for generating adipose tissue lysing, such as, for example by cavitation, and arranged such that at the focal point where they overlap each other, the power density is such as to generate cavitation, or any other selected effect, which will cause lysing in the tissue.

Reference is now made to FIG. 11, which shows an exemplary spherical cap-shaped unitary transducer, with 160 segmented transducers thereon, which may be advantageously formed by one of the methods mentioned hereinabove, using segmented electrodes. The transducer segments are arranged over the surface of the transducer head such that they can be fired in any predetermined order designed for the treatment at hand. The optimal distribution is such as to achieve maximal beam steering range and maximum achievable pressure at each focal point, with minimum side-lobe level, while using the minimum number of transducer segments.

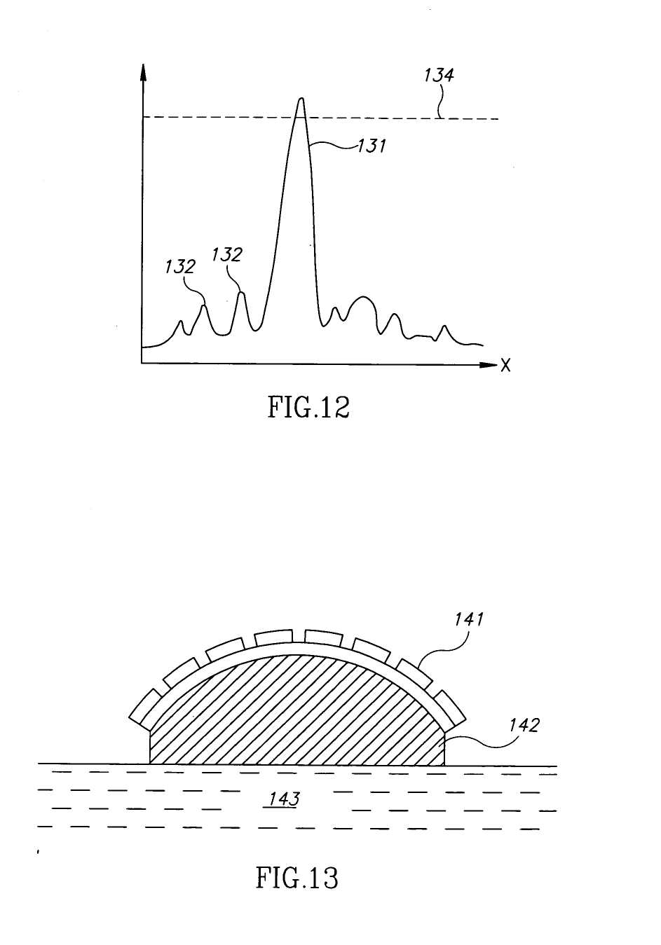

FIG. 12 illustrates a graph of the spatial intensity profile of the ultrasound energy impinging on target, for a typical arrangement of transducer segments. The profile has a main lobe 131 and side lobes 132, as is common for any beamed transmission. It is known that when ultrasound impinges on body tissue, pain is felt by the subject when the intensity exceeds a certain threshold, marked in the graph as 134. Furthermore, the existence of a large proportion of the energy in the side lobes is inefficient for two reasons—(i) since there is a limited amount of power generated by the transducer head, any power spread out in the side lobes reduces the power available for the main lobe, and (ii) it deposits energy in the region surrounding the target, which is below the level at which any therapeutic effect is generated, but it does produce cumulative background heat. Therefore, it is important to generate a beam propagation profile such that the main lobe has the maximum possible concentration of power, while not exceeding the pain threshold level. These requirements translate in practice to a broader main lobe having a gentler rise to its peak, a peak intensity preferably not exceeding the estimated pain threshold, and minimal side lobes. Such a tailored profile can be readily achieved using transducer phased arrays, according to the various embodiments of the present disclosure.

In order to obtain the best arrangement of placement and firing of the segments, a placement algorithm has been developed. The problem to be solved is that if the segments are placed with maximum and ordered coverage of the transducer surface, there is optimum transducer output, but the interference of beams from the ordered segments generates Fresnel zones, which give rise to the side lobes at the focal plane. A completely random placement and firing of the segments will reduce any constructive interference effects, and will thus suppress the side-lobes, as desired. However, there will then be reduced flux output from the transducer. A semi-random placement of the segments, as determined by the algorithm developed for optimizing the segment positions, provides optimum coverage in conjunction with minimum side-lobes. According to one exemplary embodiment, the algorithm operates by taking orderly groups of segments, and placing them randomly over the surface. A criterion combining the levels of transducer output and the level of side-lobe suppression is built, and the placement is varied iteratively to optimize this criterion. The mathematical background for performing this iteration is shown below, but it is to be understood that the invention for optimizing segment placement is not meant to be limited by this particular algorithm, but others can equally well be used, so long as the criterion for optimal coverage is properly defined.

The algorithm is calculated for the placement of circular elements on a spherical segment.

The spherical cap (concave) is specified by following parameters:

1. Curvature radius F;

2. Half-aperture angle θ₀;

3. Hole half-aperture angle θₕ;

Given the cup parameters, the segment area is calculated as:

S₀=2πF²(cos θₕ−cos θ₀) (1)

The radius r of each of N elements, which have to be placed on the cup, can be calculated as:

$$r = \sqrt{\frac{α {S}_{0}}{π N}} \qquad ( 2 )$$

Here α is a coefficient of the segment area coverage with the elements.

Given r, which is calculated with (1), (2), the placing of the elements is fulfilled as follows. Every point at the cup is specified by two spherical coordinates: the polar angle θε[θₕ, θ₀] and the azimuth angle φε[0,2π]. The region in which the elements' centers can be placed is restricted with regards to θ as θε[θₕ+θᵣ, θ₀−θᵣ], =θᵣ=arcsin(r/F). The standard randomizer program is run sequentially to generate pseudo-random numbers which are uniformly distributed with respect to φ and θ within the chosen ranges. The first generated pair (θ₁, φ₁) is stored. The number of successfully accommodated elements n is set to 1. Then the newly generated pair (θ, φ) is checked on whether or not it satisfies the condition:

$${min}_{i = 1} {d}_{i}^{2} > 4 {r}^{2} , {d}_{i}^{2} = {( x - {x}_{i} )}^{2} + {( y - {y}_{i} )}^{2} + {( z - {z}_{i} )}^{2} , \qquad ( 3 )$$

where x=F sin θ cos φ, y=F sin θ sin φ, z=F(1−cos θ). The axis z coincides with the cup acoustic axis. The coordinate origin is put on the top (apex) of the concave. If the condition (3) is satisfied, then the found pair is stored as (θₙ₊₁, φₙ₊₁) and the number n growths: n→n+1. The algorithm runs while n<N and number of undertaken trials does not exceed the allowed one (10⁶ in our implementation).

Apparently, the algorithm fails if the tried coefficient of the segment area coverage exceeds some maximally allowable value, which depends on the cup parameters and on the number of elements to place. The actual value can be found with the binary search. Namely, the appropriate region of the coefficient search [αₘᵢₙ, αₘₐₓ] is firstly initialized as [0,1]. The placing is fulfilled at α=αₘᵢₙ=0 and the results are stored. Then the algorithm proceeds as follows.
1. The accommodation trial is undertaken at α=(αₘᵢₙ+αₘᵢₙ)/2;
2. If the attempt is successful then a) the results are stored, b) αₘᵢₙ=α, else αₘₐₓ=α. After that the first item is repeated.
   The algorithm is run while the interval (αₘₐₓ−αₘᵢₙ) exceeds some threshold (0.01 in our implementation).

The described above straightforward procedure results in random placing of N elements. However the coefficient of the segment area coverage appears to be significantly smaller than the one which can be achieved with a method of regular element placing. This shortcoming may be overcome with the following post-processing procedure.

The post-processing algorithm employs the connection between the specified above global Cartesian coordinate system and the local spherical coordinate system, which is associated with the apex of i-th element. The top (apex) of i-th element is specified by the pair (θᵢₚ φᵢ) of the global spherical coordinate system associated with the cup apex. Given i-th local spherical coordinates (θ′, φ′) of some point at the cup, the global Cartesian coordinates of the point are calculated as:

x(θ′, φ′; θᵢ, ᵢ)=F(sin θ′ cos θᵢ cos φᵢ cos φ′−sin θ sin φ₁ sin φ′+cos θ′ sin θᵢ cos φᵢ)

y(θ′, φ′; θᵢ, φᵢ)=F(sin θ cos θᵢ sin φᵢ cos φ′+sin θ′ cos φᵢ sin φ′+cos θ′ sin θᵢ sin φᵢ)

z(θ′, φ′; θᵢ, φᵢ)=F(1−cos θ′ cos θᵢ+sin θ′ sin θᵢ cos φ′) (4)

The post-processing procedure is specified by choosing some polar angle θ′ which must be much smaller than the ratio r/F and by the dimension M of the azimuthal grid

φ^{k}=kΔφ, Δφ=2π/M, k=0,1, . . . , M−1

In our implementation, those parameters are θ′=10⁻³ r/F, M=100. The procedure is initialized by the calculation and storage of the minimal squared Euclidian distance

$${d}_{min}^{2} = {min}_{i , j} {d}_{ij}^{2} , {d}_{ij}^{2} = {( {x}_{i} - {x}_{j} )}^{2} + {( {y}_{i} - {y}_{j} )}^{2} + {( {z}_{i} - {z}_{j} )}^{2} \qquad ( 5 )$$

Here (xᵢ,yᵢ,zᵢ) are Cartesian coordinates of i-th element's apex. After the initialization the following steps are sequentially fulfilled for each of earlier placed N elements:

1. The apex of i-th element is shifted taking Cartesian coordinates

(xᵢ^{k}, yᵢ^{k}, zᵢ^{k})=(x(θ′, kΔφ, θᵢ, φᵢ), y(θ′, k Δφ; θᵢ, φᵢ), z(θ′, kΔφ; θᵢ, φᵢ)), k=0,1, . . . , M−1

2. The squared Euclidian distances matrix

dᵢⱼ^{k2}=(xᵢ^{k}−xⱼ)²+(yᵢ^{k}−yⱼ)²+(zᵢ^{k}−zⱼ)²

is calculated for every j≠i. The matrix is supplemented by the vector d_{iM}^{k2}, which is power of two of a double minimal distance from the k-th location of i-th element to the cup border.
3. The index k₀ which satisfies the condition

$${d}_{i}^{{k}_{0} 2} = {min}_{j} {d}_{ij}^{{k}_{0} 2} \geq {min}_{j} {d}_{ij}^{k 2}$$

is sought.
4. If the squared distance dᵢ^{k}^{ₒ}² is bigger than dᵢ²=minⱼ dᵢⱼ² then the global spherical coordinates of i-th element are updated as:

θᵢ=arccos(1−zᵢ^{k}^{ₒ}/F), φᵢ=arctan(yᵢ^{k}^{ₒ}/xᵢ^{k}^{ₒ})

After carrying out steps 1-4 for all i=1,2, . . . , N the minimal squared distance (5) is calculated again and compared with the stored one. If the difference of the two distances appears to be less than some threshold (in our implementation 10⁻⁴r²) then the post-processing procedure is stopped, otherwise the new minimal squared distance is stored and the steps 1-4 are repeated.

After the post-processing, the element radius can be set as r=\sqrt{dₘᵢₙ²}/2. The described procedure enables significant enlargement of the coverage coefficient making it comparable with the one which can be obtained with some method of a regular element placing.

As previously mentioned, the phenomenon known as Time Reversal may be used for increasing the efficacy of the treatment. Time reversal can be generated by mounting the transducer on a resonator (a device, which exhibits acoustic resonance behavior such that it may oscillate at some frequencies with greater amplitude than at other frequencies), sensing the ultrasound pulses transmitted into the tissue, time-reversing the pulses electronically, and applying the time reversed pulses to the transducer driver. Reference is now made to FIG. 13 which shows an array of segmented transducers 141, according to an embodiment of the present disclosure, mounted on a resonator 142 for generating time reversed operation of ultrasound treatment of a subject's tissue 143. If several transducers are mounted on a single resonator, the directionality of the individual transducers is generally lost. On the other hand, if individual transducers, or groups of transducers, are mounted on several resonators, it is possible to maintain directionality and to operate a phased transducer array with time reversal. The groups of transducers could then be arrays formed according to the embodiments of the present disclosure.

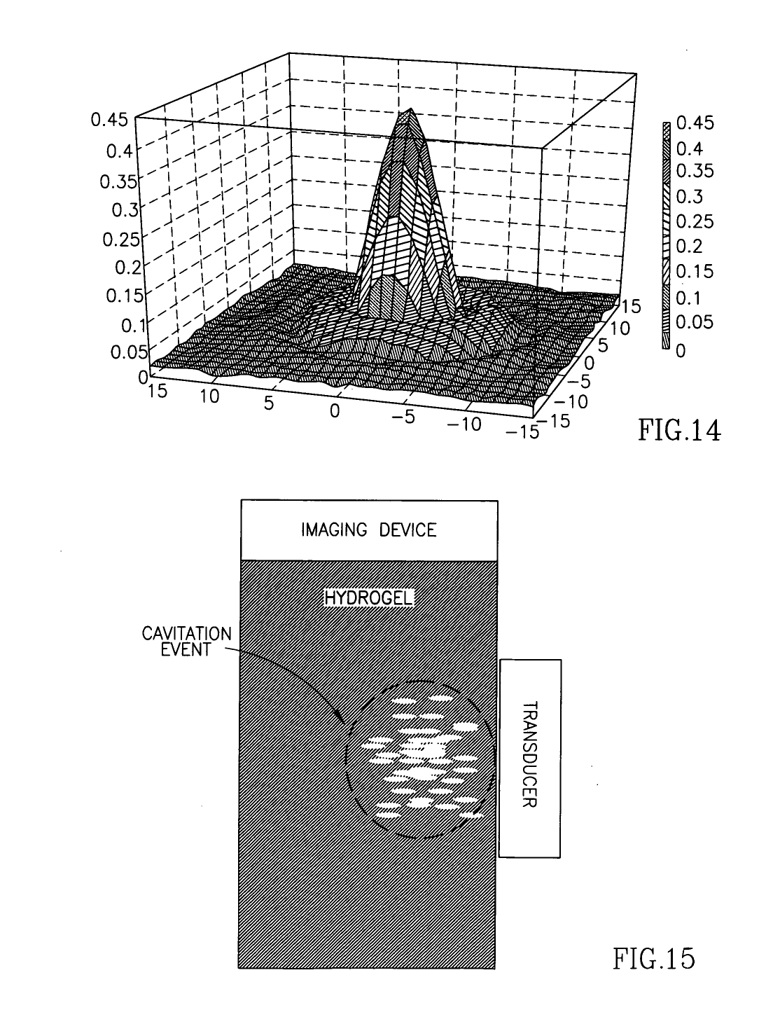

According to some embodiments, and further to what is mentioned hereinabove, a transducer may be operative such that by selection and/or use of appropriate parameters, a selective formation of an effect, such as, for example, cavitation in a target tissue, may be achieved. For example, by selecting appropriate parameters, forming of cavitation in/on/at an adipose and/or cellulite tissue may be achieved, while adjoining and/or near and/or surrounding tissues (such as blood, muscle, nerve, connective or other tissues) may not be affected. Therefore, a transducer, with one or more transducing elements, as described hereinabove, may be constructed and operated with such parameters that maximal selectivity of its effect is achieved. For example, a transducer, comprising one or more transducing elements (zones), as described hereinabove may operate with the following exemplary parameters listed below to obtain selective effect on adipose/cellulite tissues and not on neighboring tissues. For simplicity, the parameters of a transducer with one transducing element (zone) are described below in the section Aspects of operation of an ultrasonic transducer (Table 2). However, it will be evident to one of skill in the art that two or more transducing zones may be similarly operative, according to various embodiments of this disclosure. For example, for one transducing zone operating at an operating frequency in the range of about 0.19 to 0.21 MHz at a pulse operating mode, with a pulse duration in the range of about 1.8 to 2.2 milliseconds (ms), with a pulse repetition period in the range of 34 to 46 ms, with exposure time of about 2.85 to 3.15 seconds per node, the following measurements are obtained: I_{SPTA} of, about 16.0 to 20 W/cm²; I_{SPPA} of, about 320 to 400 W/cm²; Pr, in the focus, of about 3.5 to 4.5 (MPa), MI (MPa/(MHz)1/2) in the focus, of about 8 to 10 (MPa/(MHz)^{1/2}); Focus depth of about 12 to 16 mm; Focal Area diameter (in the focal plane) of about 5 to 7 mm. The results show that the transducer (transducing zone) produces focused ultrasound with the maximum pressure value at the depth of 14 mm. The ratio of the acoustic pressure in the focus to the maximal pressure on the surface (skin) is in the range 3.5-4.0, which further ensures safety of the treatment. Results of testing the effects thus produced by transducer operative with the listed parameters are further detailed in Aspects 1 and 2 (FIG. 14 and 15, respectively).

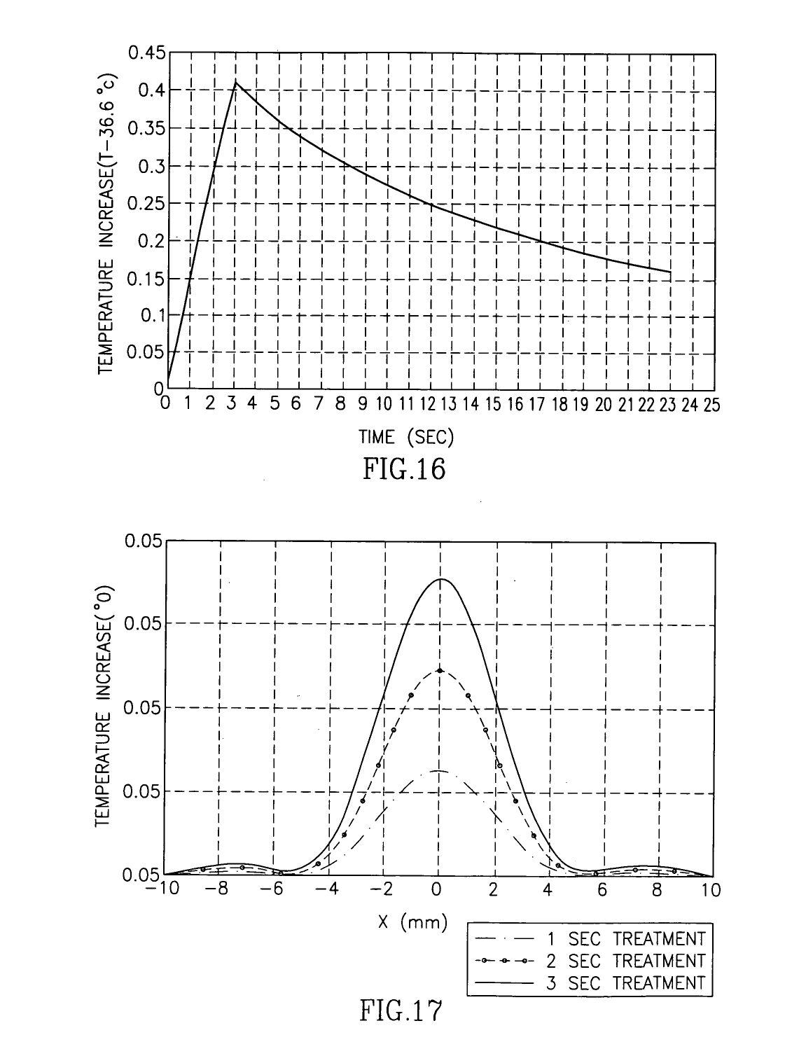

Comparing the results thus obtained from a transducing element operating with the parameters essentially as listed hereinabove, with those listed in Table 2 demonstrate the following points: 1. Although the pressure values in the focus are in the range of the diagnostic ultrasound, the I_{SPTA} values are higher. In addition, calculated MI value (which characterizes the likelihood of mechanical damage) is averaged at about 9.0, which is significantly above the maximal allowed value 1.9 for diagnostic equipment and, as mentioned above, is in the range of the cavitation threshold in tissues. This means that the transducer element is selectively adapted to mechanically destruct fat cells. 2. The calculated Pᵣ and I_{SPTA} values are much lower than those for HIFU applications listed in Table 1 (which include thermal, histotripsy and haemostasis procedures). A pulsed operation mode (with a duty cycle of about 5%), a comparatively low Pr and ISPTA values, and short exposure time per node practically exclude any noticeable heating that may be caused by the transducer. As detailed in Aspects 3 and 4 (FIG. 16 and 17, respectively), calculations of the spatial temperature rise distribution performed using the Pennes bio-heat equation (1) show that it does not exceed 0.5° C. in the focus area.

Therefore, in view of the results obtained from the operating parameters presented hereinabove, it may be stated that the transducer is not operative under the “classical” definition of HIFU. Rather, the transducer is operative in the Mid Intensity focused ultrasound (MIFU) and/or the low intensity focused ultrasound (LIFU). In spite of this definition, the treatment rendered by use should have the same cumulative effects as those of conventional HIFU, yet without the above-delineated disadvantages of conventional HIFU treatment.

The results of several preclinical and clinical studies performed for treatments using essentially the operating parameters listed hereinabove and in Table 2 demonstrate that such treatments produce safe and selective mechanical lysis of fat cells.

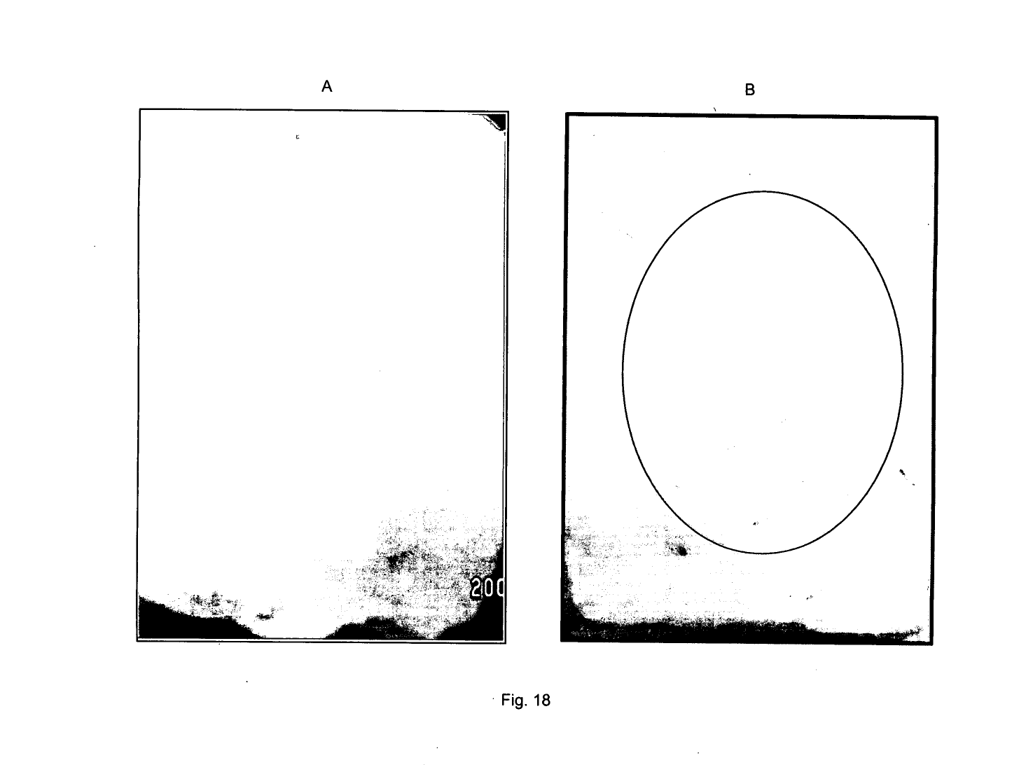

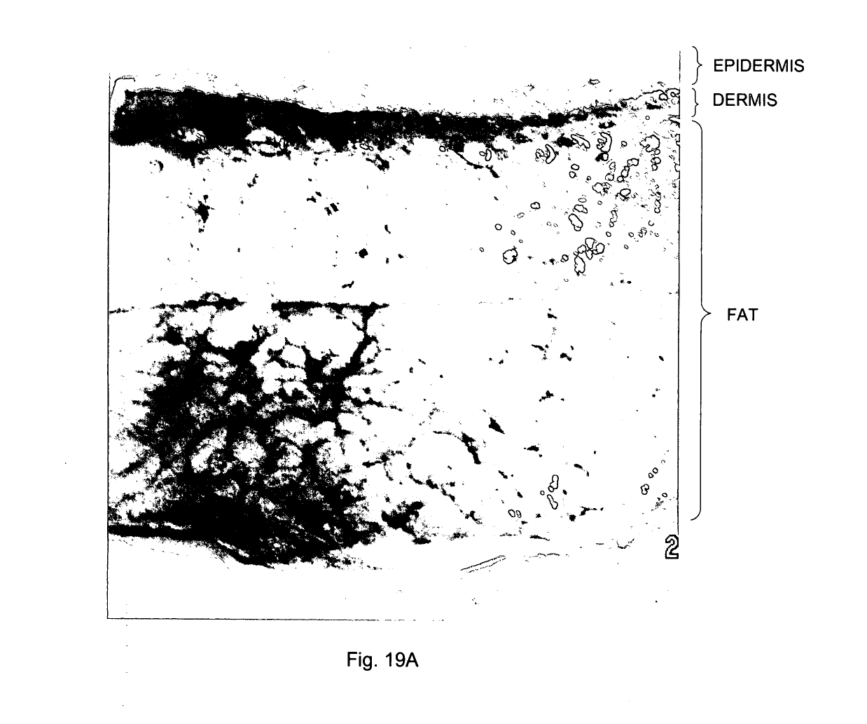

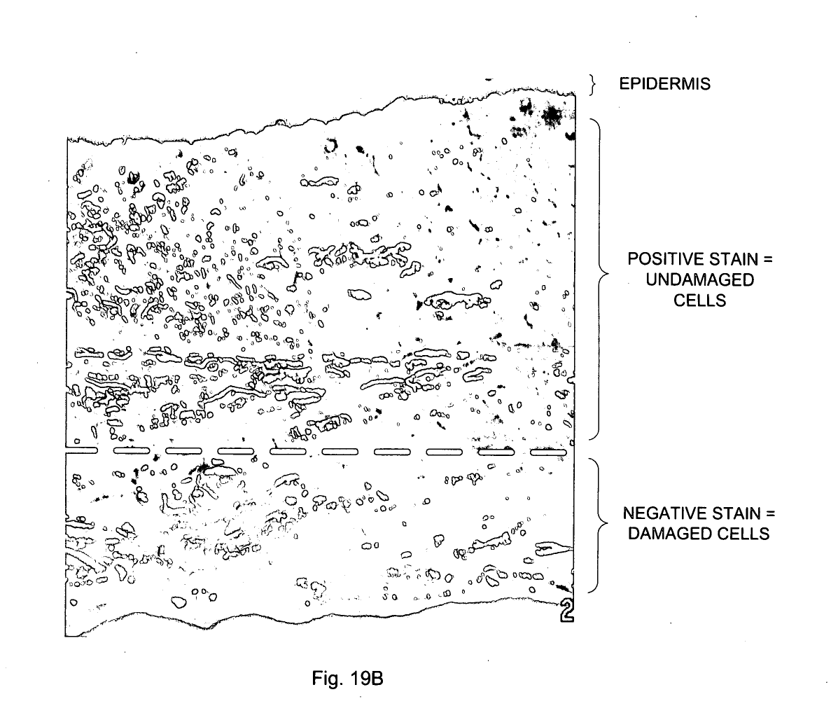

For example, some of the pre-clinical studies are based on the porcine model, which is considered as an accepted and frequently used model for studies in liposuction and skin safety, since the fat and skin of this animal have been demonstrated to be comparable to human fat and skin. Furthermore, large animal models are desired for providing an adequate size for full contact of the transducer with the skin and sufficient thickness of fat to ensure that the focal area will be within the subcutaneous fat layer. The pre-clinical studies on the porcine model may be performed at two levels: Ex-vivo—wherein the treatments and evaluations are performed on excised fat tissue. In such experiments, preliminary feasibility is enabled in short time frames; In-vivo—the treatments are performed on live pigs, which may enable the evaluation of the ultrasound effect in a living body. In this case, the systemic physiological processes such as blood flow, enzymatic reactions, and the like may be involved. Results of several exemplary studies, which demonstrate the safety and selectivity of treatments, are presented in Aspect 5 (FIGS. 18-FIG. 20F) below.

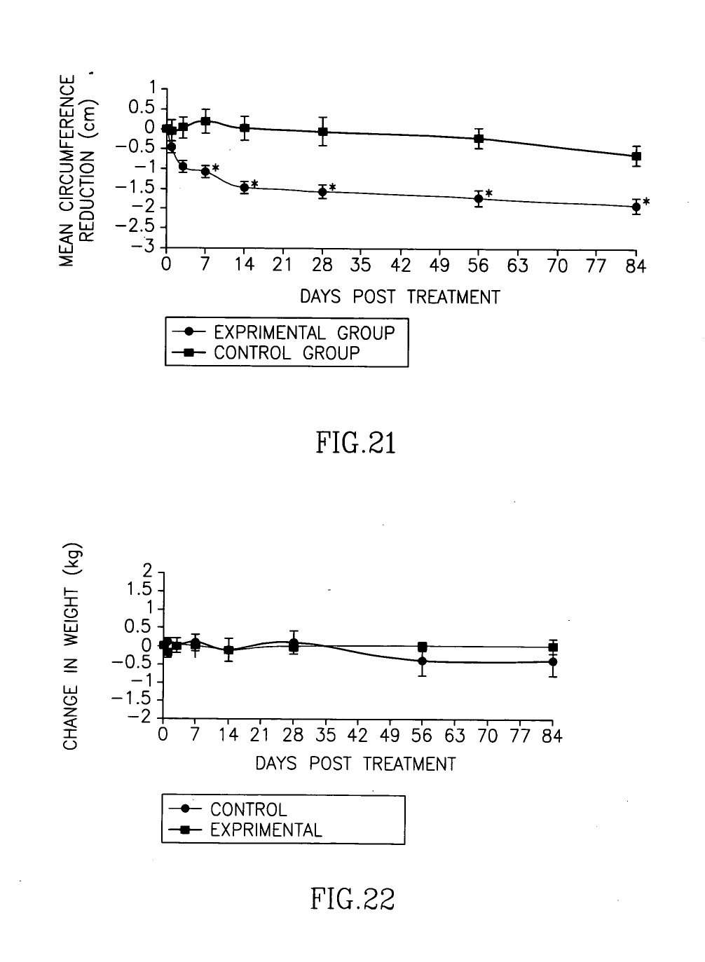

According to additional examples, the safety and efficacy of the body contouring ultrasonic treatment, with the parameters essentially as listed in Table 2, was further assessed and confirmed in a multicenter clinical trial conducted at five centers (two in the United States, one in the United Kingdom, and two in Japan). Briefly, one hundred sixty-four healthy volunteers were enrolled in this prospective comparative study, of which 137 participants were assigned to the experimental (treated) group and 27 participants were assigned to the control (untreated) group. Follow up visits for both experimental and control groups were scheduled on days 1, 3, 7, 14, 28, 56 and 84. The participants of the experimental group received a single treatment in the abdomen, thighs or flanks. The results of these experiments are summarized herein below in aspect 6 (FIGS. 21-22 and Table 3). The results demonstrate that the effects observed after treatment (such as, for example, reduction in circumference) are attributed to the treatment. The results further demonstrate that no clinically significant changes were observed in laboratory testing, pulse oximetry and liver ultrasound of participants of trials.

Additionally, the effect of multiple treatments as detailed above herein was evaluated in a prospective study conducted on 39 healthy patients. All participants underwent three treatments, at 1-month intervals, and were followed for 1 month after the last treatment. Efficacy was determined by change in fat thickness, assessed by ultrasound measurements, and by circumference measurements. The results, which are detailed in Aspect 7, illustrate that a significant reduction in subcutaneous fat thickness within the treated area and circumference reduction was observed with all patients.

Although the ultrasound phased array system of the present disclosure has been described in terms of its use in fat removal, it is to be understood that the advantages of the use of such an ultrasound phased array system to generate an accurate and controlled high intensity focused beam of acoustic energy can be equally well applied for therapeutic treatment of various other medical conditions, including the non-invasive destruction of growths by tissue ablation or destruction.

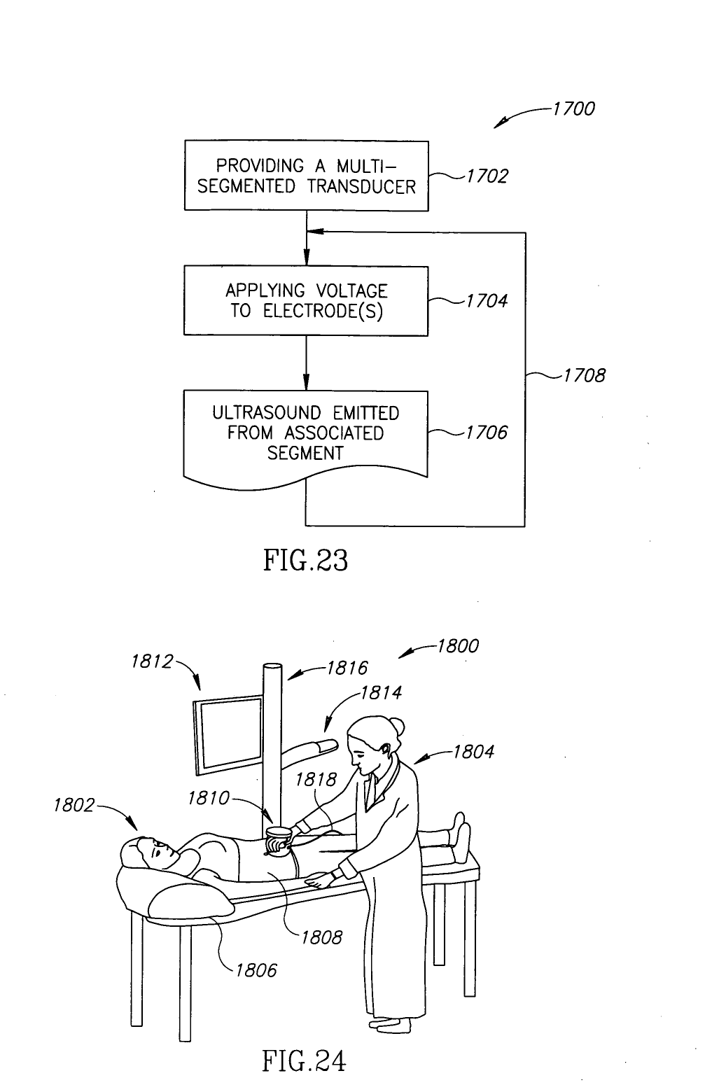

Reference is now made to FIG. 23, which shows a flow chart 1700 illustrating a method for generating focused ultrasound energy for lysing of adipose tissues, according to an embodiment. In a block 1702, a multi-segmented transducer (also referred to as a “transducer array”) is provided and positioned at a desired location. In a body contouring position, the transducer may be positioned substantially over a portion of a patient's body, above an approximate area of treatment.

In a block 1704, voltage is applied to at least one electrode and/or electrode element of the transducer. A plurality of electrode elements may be associated with a plurality of distinct segments of the transducer. Voltage may therefore be applied simultaneously and/or sequentially to one or more electrode elements, where at least some of the electrode elements may be associated with different segments.

In a block 1706, the applied voltage excites vibrations in one or more segments of the transducer, where each segment may be associated with one or more of the electrode elements. The vibrations induce emitting of ultrasonic waves from the piezoelectric material forming the transducer.

The application of voltage in block 1704, followed by the emitting of ultrasound in block 1706, may be repeated 1708 a desired number of times.

In an embodiment, a multi-segmented transducer is used in a body contouring procedure—a procedure wherein adipose tissues are destroyed for reshaping and essentially enhancing the appearance of a human body.

Reference is now made to FIG. 24, which shows an exemplary treatment 1800 of a patient 1802 by a caregiver 1804. Caregiver 1804 may be, for example, a physician, a nurse and/or any other person legally and/or physically competent to perform a body contouring procedure involving non-invasive adipose tissue destruction. Patient 1802 optionally lies on a bed 1806 throughout treatment 1800.

Caregiver 1804 may hold a transducer unit 1810 against an area of patient's 1802 body where destruction of adipose tissues is desired. For example, transducer unit 1810 may be held against the patient's 1802 abdomen 1808. Transducer unit 1810 may comprise one or more multi-segmented transducers. Transducer unit 1810 may be connected by at least one wire 1818 to a controller (not shown) and/or to a power source (not shown).

Optionally, a user interface is displayed on a monitor 1812, which may be functionally affixed to a rack, such as pillar 1816. A transducer unit 1810 storage ledge 1814 may be provided on pillar 1816 or elsewhere.

Body contouring may be performed by emitting one or more ultrasonic pulses from transducer unit 1810 while it is held against a certain area of the patient's 1802 body. Then, transducer unit 1810 is optionally re-positioned above one or more additional areas and the emitting is repeated. Each position of transducer unit 1810 may be referred to as a “node”. A single body contouring treatment may include treating a plurality of nodes.

### Aspects of Operation of an Ultrasonic Transducer

Listed in Table 2 are operating parameters of a transducer, the operating aspects of which are discussed hereinbelow.

TABLE 2

| Operating Parameters | Value |
|---|---|
| Operating Frequency (MHz) | 0.2 ± 0.03 |
| Operating Modes | Pulsed (tone bursts) |
| Pulse Duration (ms) | 2.0 ± 15% |
| Pulse Repetition Period (ms) | 40 ± 15% |
| Exposure time per node (s) | 3.0 ± 5% |
| ISPTA (W/Cm²) | 18.0 ± 10% |
| ISPPA (W/cm²) | 360.0 ± 10% |
| Pᵣ (MPa), in the focus | 4.0 ± 0.5 |
| MI (MPa/(MHz)^{1/2}), in the focus | 9.0 ± 1.0 |
| Focus depth (mm) | 14.0 ± 2.0 |
| Focal Area diameter (in the focal | 6.0 ± 1.0 |
| plane), mm | |

Aspect 1—Acoustic field distribution in the focal plane of a transducer, measured in water with a hydrophone.
Shown in FIG. 14 is the acoustic field distribution in the focal plane of the transducer, measured in water with a hydrophone. The results show the distribution of the peak pressure (in units of MPa) in the focal plane of the transducer.
Aspect 2—A cavitation effect produced by the transducer in hydrogel and visualized by an imaging device (ultrasonic imager).
Shown in FIG. 15, a cavitation effect produced by the transducer in hydrogel and visualized by an ultrasound imager. The cavitation effect is demonstrated by white ellipses.
Aspect 3—Temperature variations with time in the focus.
Shown in FIG. 16, a graph illustrating temperature variation (in Celsius degrees) with time (Sec) in the focus of the ultrasound.
Aspect 4—Radial temperature increase distribution in the focal plane.
Shown in FIG. 17, a graph illustrating the distribution (measured in mm) of radial temperature increase (in Celsius degrees) after 1 second, 2 second and three second treatments, in the focal plane.
Aspect 5—Ex-vivo and in-vivo pre-clinical studies on the porcine model.
The studies which are presented in aspect 5 utilize the porcine model, which is considered as an accepted and frequently used model for studies in liposuction and skin safety, since the fat and skin of this animal have been demonstrated to be comparable to human fat and skin. Several experimental techniques, which are well known in the art are utilized in those examples. Briefly, the techniques may include:

1. Histology evaluations: in order to evaluate the ultrasound effect on subcutaneous fat, along with safety and selectivity considerations, various histology techniques and cell viability assays are performed routinely. (Results of various histology evaluations are shown in the relevant examplery figures in gray-scale).
i. H&E—The hematoxylin and eosin stain (designated as H&E) is a combination of two dyes: the basic dye hematoxylin, and the alcohol-based synthetic material, eosin. H&E is a structural stain, primarily providing morphological information. The appearance of a tissue with H&E is regarded as an “actual” one, and it may be used as a basis for comparison when special stains are applied to reveal some other aspect of the tissue's structure or chemistry. The staining reaction is clearly stronger in some parts of the tissue and cells than in others, allowing identification of the details. H&E may be used on both paraffin-embedded tissues and frozen sections (described below).
ii. Masson's Trichrome—This stain enables easy distinction between extensive collagenous and elastic fibers of the connective tissues, the walls of veins and arteries (usually stained in blue) and the cytoplasm of cells (usually shaded in red). In the relevant exemplary figures shown below, the staining and differential staining are shown in gray-scale.
iii. LDH-activity staining—Lactate dehydrogenase (LDH) is an enzyme which catalyses the conversion of lactate to pyruvate during the cellular respiration process. The LDH-activity stain is used to indicate and discriminate between viable and non-viable areas in the tissue following various ultrasound treatments and to provide better understanding of how the ultrasonic treatment affects the subcutaneous fat. A blue dye (shown in the relevant exemplary figures in gray-scale) is formed within live cells. Regions that would not be stained within the sample mean those cells are harmed.

2. Sectioning techniques: the most common technique to cut fixed tissues is the paraffin-embedded tissue (PET) method. Tissues are commonly embedded in a solid medium to facilitate sectioning. To obtain thin sections in the microtome, tissues must be infiltrated after fixation with embedding substances that impart a rigid consistency to the tissue. The most common embedding material for light microscopy is paraffin. Although this technique enables high quality discrimination between various compartments within the tissue, the technique is not optimal for fatty materials such as adipose tissue. Formalin fixation of hydrophobic tissues (a crucial step prior to the embedding procedure) demands a long incubation during of at least 72 hours. During this period, the harvested tissue is under stress, and autolysis pathways such as lysosomal enzymatic activity occur, a phenomenon that may lead to artifact ruptures and spontaneous lyses. Since the effect of present ultrasonic treatment in the adipose tissue may be visualized as a cluster of small holes (1 mm each) and the adipose tissue is considered as soft and hydrophobic, it might occur that the unaffected surrounded tissue collapses into the small holes. Therefore, a technique of snap freezing of the tissue in liquid nitrogen could be an appropriate alternative for the procedure of the tissue embedding. In snap freezing, the tissue is rapidly frozen rock-hard and held at liquid nitrogen temperatures. In this way, the tissue texture is kept “as is” with no artifact alterations. Then, it is cut in a special refrigerated microtome called a cryostat just as easily as embedded specimens are. This technique enables freezing of all cellular enzymatic/metabolic activities with no need for using water-based fixatives.

Following the ultrasonic treatment, the fat and overlying skin (4 mm thick) of a mature swine were dissected immediately after animal sacrifice. The histological evaluation was performed using the H&E and/or Masson's Trichrome staining on frozen sections and paraffin-embedded tissues. In addition, LDH-activity stain was used to indicate and discriminate between viable and non-viable areas in the tissue. FIG. 18 demonstrates gray scale pictorial macroscopic histological evaluation of the effect of ultrasonic treatment on the swine adipose tissue. FIG. 18A demonstrates untreated tissue, while FIG. 1 8B demonstrates ultrasonic treated tissue. As shown in FIG. 18B, the ultrasonic treatment result in a cluster (a circle) of small holes in different sizes (up to 1.5 mm each) within the adipose tissue.

FIG. 19 demonstrates gray scale pictorial LDH staining evaluation of an ultrasonic treatment on the swine adipose tissue. FIG. 19A demonstrates untreated tissue, while FIG. 19B demonstrates ultrasonic treated tissue. Various tissue layers are indicated (Epidermis and dermis skin tissue) and fat tissue. The results show that while LDH-activity stain is performed on both treated (FIG. 19B) and untreated tissues (FIG. 19A), the indication for cellular damage (designated arrows) is seen only in the treated tissue, 14 mm under the surface, where the ultrasound energy is focused.

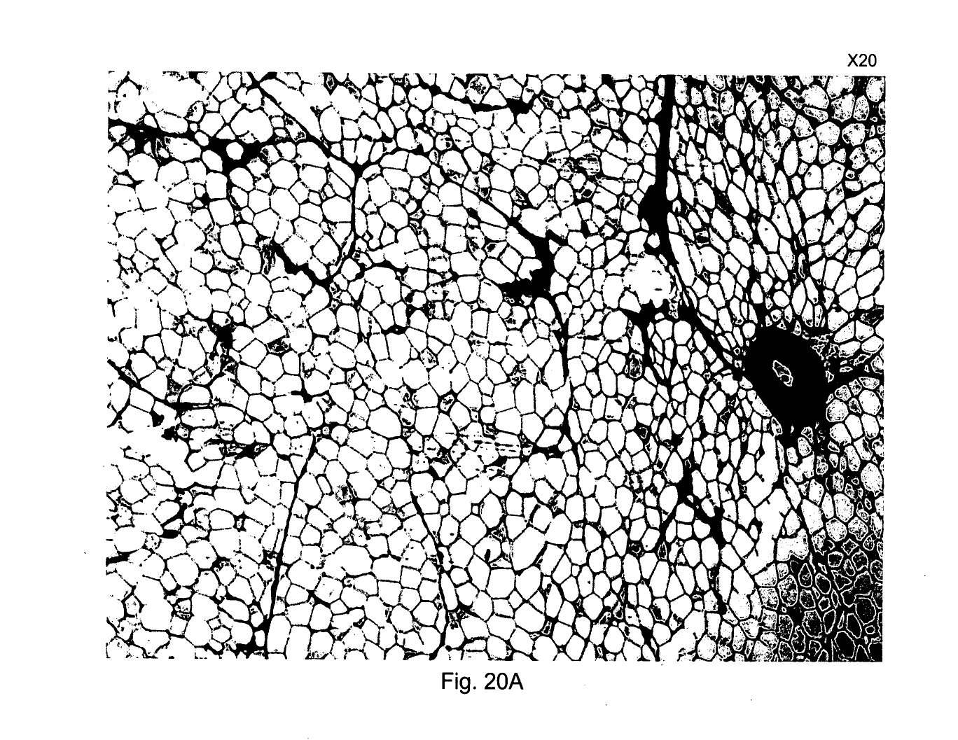

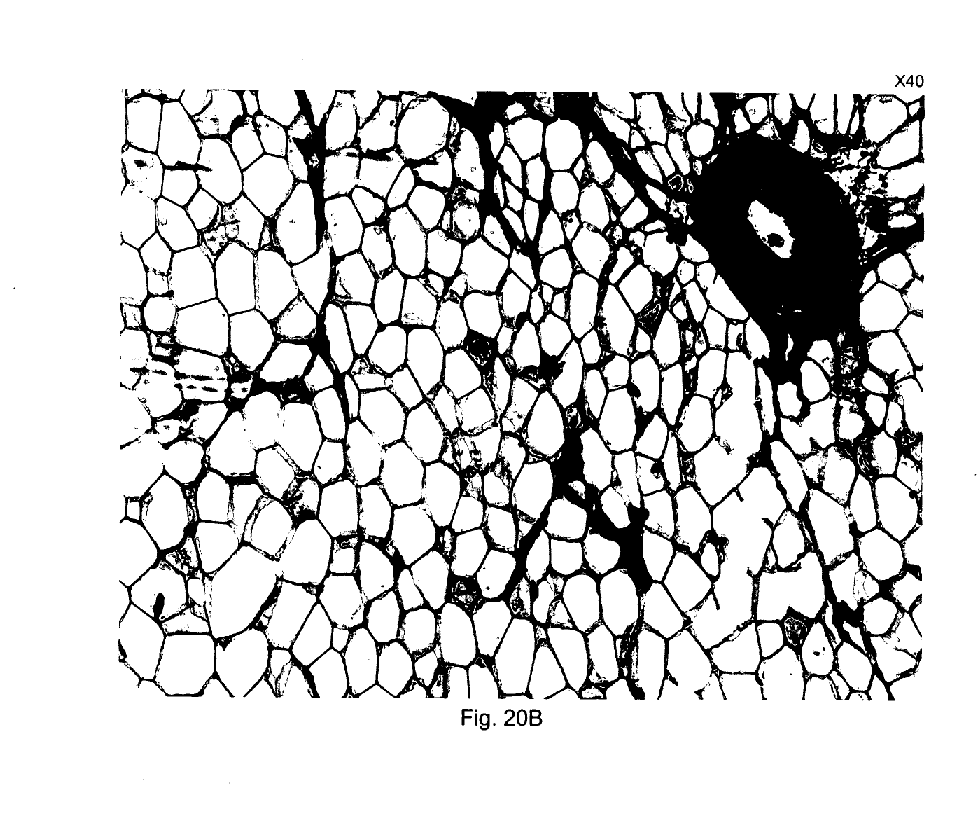

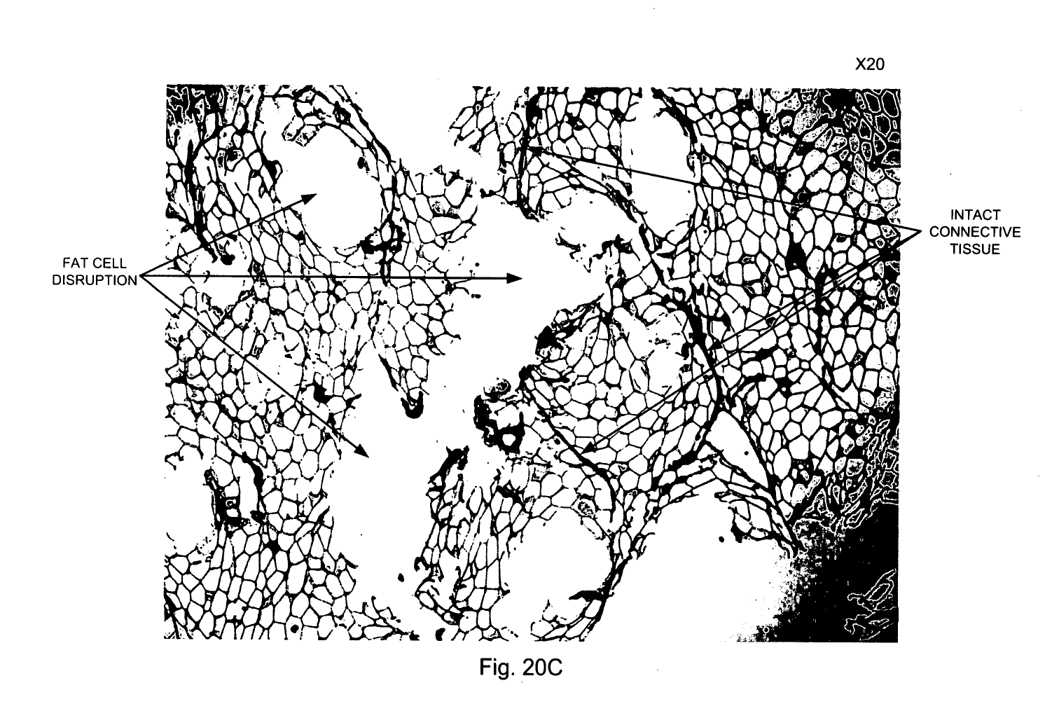

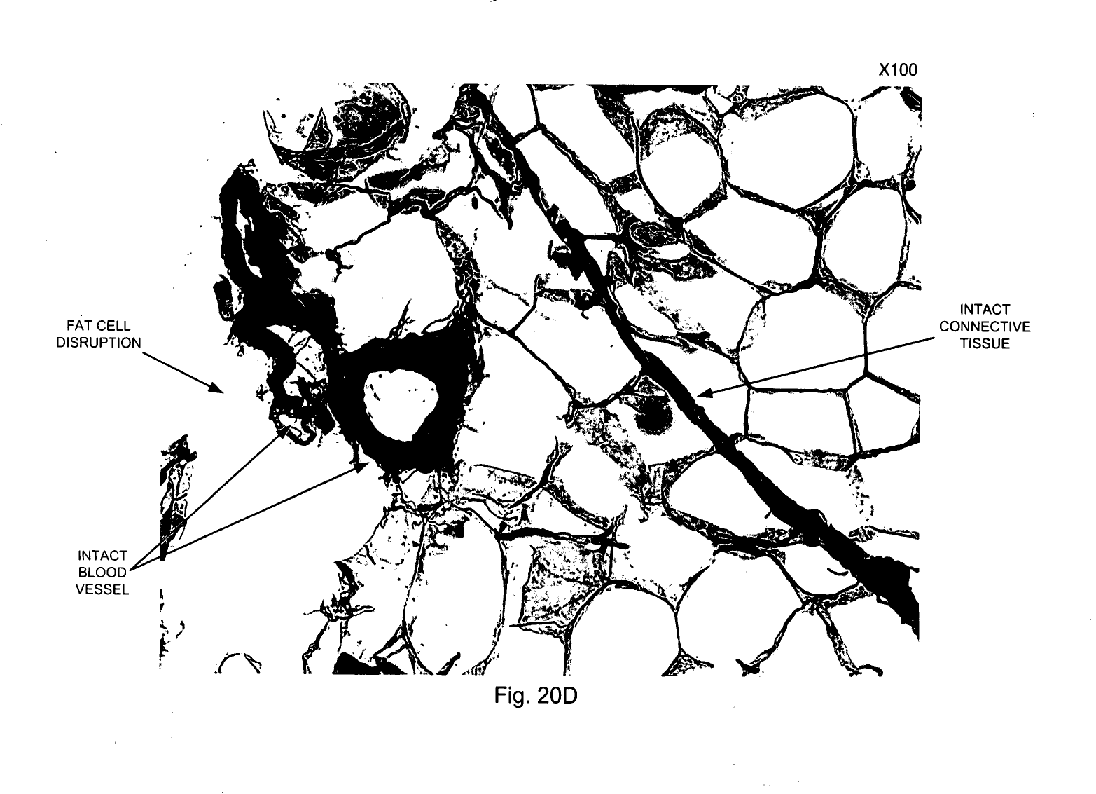

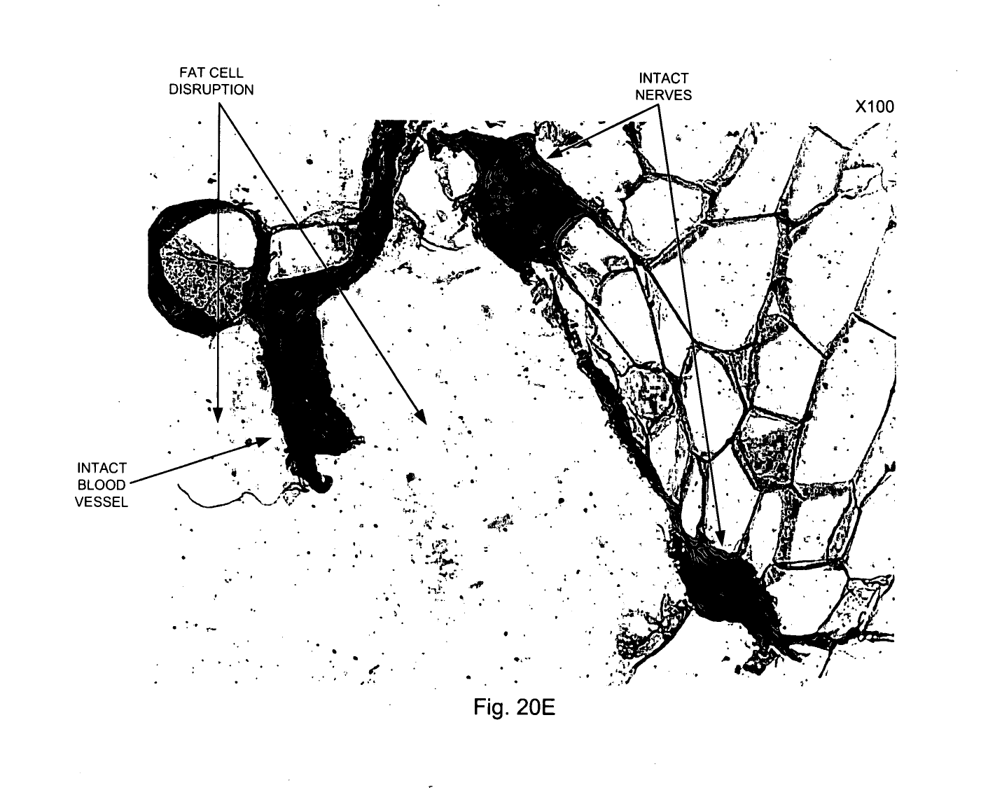

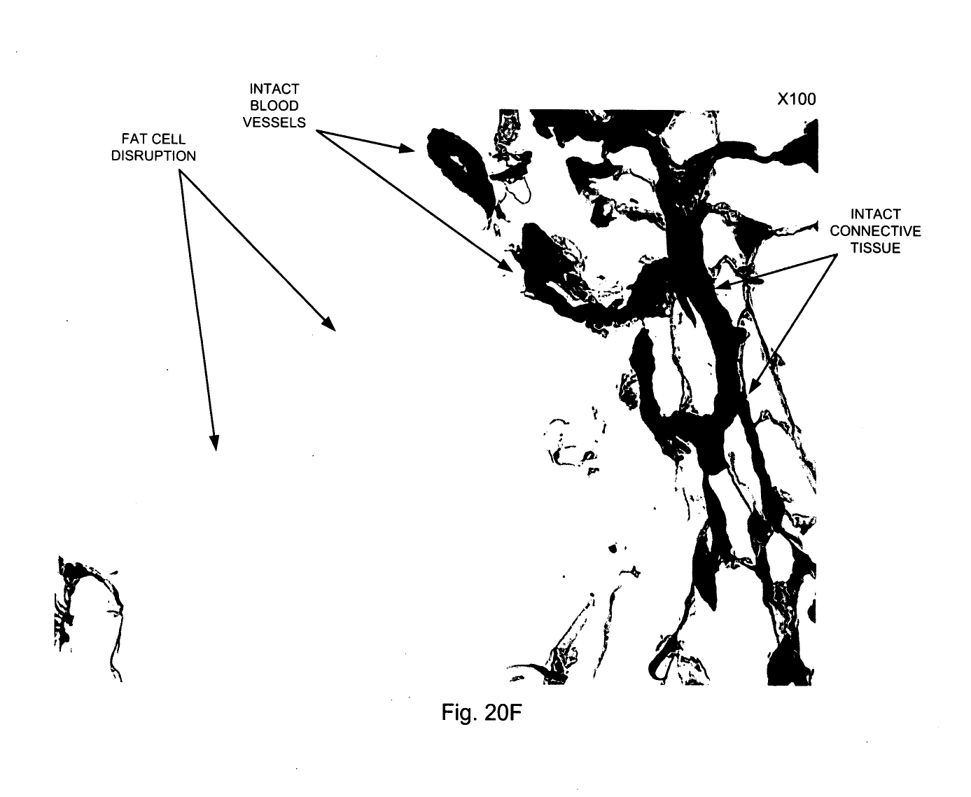

FIG. 20 demonstrates gray scale pictorial microscopic histological evaluation of swine tissues. Shown in FIGS. 20A-B is an untreated tissue. Shown in FIGS. 20C-F is treated tissue. As shown in FIG. 20, while intact fat cells are observed in the untreated control (FIG. 20A and 20B), fat damage is detected in the ultrasound-treated samples (FIG. 20C-F). The fat damage (such as adipocyte lysis) may be observed as loss of membranes of adjacent cells, which creates holes in different sizes. The ultrasonic treatment is selective as clearly demonstrated in FIGS. 20C-F, which show that while adipocytes disruption is observed (designated arrows), other tissues, such as connective tissue (designated arrows in FIG. 20C and FIG. 20D), blood vessels (designated arrows in FIG. 20D and FIG. 20F) or nerve tissue (designated arrows in FIG. 20E) remain intact.

Aspect 6—Clinical studies of single ultrasonic treatment, according to some embodiments.
The safety and efficacy of the ultrasonic treatment was confirmed in a multicenter clinical trial conducted at five centers (two in the United States, one in the United Kingdom, and two in Japan). One hundred sixty-four healthy volunteers were enrolled in this prospective comparative study. From them, 137 participants were assigned to the experimental (treated) group and 27 participants were assigned to the control (untreated) group. Follow up visits for both experimental and control groups were scheduled on days 1, 3, 7, 14, 28, 56 and 84. The participants of the experimental group received a single treatment in the abdomen, thighs or flanks.

A single treatment resulted in a mean circumference reduction of 1.9 cm at 12 weeks, with a response rate of 82 percent. In the control group no statistical differences were observed in the mean circumference reduction from baseline, as illustrated in FIG. 21, which illustrates a graph of mean circumference reduction (in centimeters, cm) over a time period (days) after treatment, for the experimental group and control group.

FIG. 22 illustrates a graph of change in weight (kg) over a time period (days after treatment), for the experimental group and control group. As shown in FIG. 22, weight was unchanged during the treatment and follow up period, which demonstrates that the circumference reduction (illustrated in FIG. 21) is due to the treatment only and not to weight loss.

Safety assessment of the ultrasonic treatments was performed by including laboratory testing, pulse oximetry and liver ultrasound testing on the participants of the clinical study. The laboratory testing included complete blood count, serum chemistry, fasting lipids (total cholesterol, HDL, LDL and triglycerides), liver markers and complete urinalysis during the follow up period. As shown in Table 3, below, which summarizes the safety assessment testing, no clinically significant changes have been observed.

TABLE 3

| Laboratory Study | Study Results |
|---|---|
| Pulse Oximetry | Normal |
| Liver Ultrasound | No treatment induced change |
| Urinalysis | No clinically significant changes |
| CBC | No clinically significant changes |
| PT, PTT, INR | No clinically significant changes |
| Electrolytes, BUN/Cr | No clinically significant changes |
| LFT's, Bilirubin, Albumin | No clinically significant changes |
| CPK, Calcium | No clinically significant changes |

Aspect 7—Clinical studies of multiple ultrasonic treatments, according to some embodiments.
The effect of multiple ultrasonic treatments, with the parameters essentially as described hereinabove, was evaluated in a prospective study conducted on 39 healthy patients. All participants underwent three treatments, at 1-month intervals, and were followed for 1 month after the last treatment. Areas treated were the abdomen inner and outer thighs, flanks, inner knees, and breasts (males only). Efficacy was determined by change in fat thickness, assessed by ultrasound measurements, and by circumference measurements. Weight changes were monitored to assess whether reduction in fat thickness or circumference was dependent on, or independent of, weight loss. Safety was determined by clinical findings, assays of serum triglycerides, and liver ultrasound evaluation for the presence of steatosis.

The results demonstrate that all patients showed significant reduction in subcutaneous fat thickness within the treated area. The mean reduction in fat thickness after three treatments was 2.28±0.8 cm. Circumference was reduced by a mean of 3.95±1.99 cm. Weight was unchanged during the treatment and follow up period which suggests the circumference reduction was due to the treatment, not weight loss. No adverse effects were observed.

It is appreciated by persons skilled in the art that the present disclosure is not limited by what has been particularly shown and described hereinabove. Rather the scope of the present disclosure includes both combinations and sub-combinations of various features described hereinabove as well as variations and modifications thereto which would occur to a person of skill in the art upon reading the above description and which are not in the prior art.

What we claim is:

1. A transducer array comprising:
at least one unitary piece of piezoelectric material having first and second opposing surfaces; and
a conductive layer on each of said first and second opposing surfaces, wherein at least one of said conductive layers is divided up into a plurality of electrode elements, and wherein said electrode elements, independently, are adapted to receive excitation energy of at least one of a predetermined amplitude and phase.

2. A transducer array according to claim 1, wherein the ultrasound energy emitted from said transducer array is influenced by at least one of the amplitudes and phases of the excitation energy received by the electrode elements.

3. A transducer array according to claim 1, wherein said phases are adapted to be shifted such that said ultrasound energy emitted from said transducer array is directed at an angle in accordance with the shift of said phases.

4. A transducer array according to claim 3, wherein the shift of said phases is adapted to vary as a function of time, such that said ultrasound energy executes a sweeping action in accordance with said variation of said phase shift.

5. A transducer array according to claim 2, wherein at least one of said amplitude and said phase is adapted to vary, such that a focal position of said ultrasound energy emitted from said transducer array is controlled.

6. A transducer array according to claim 2, wherein at least one of said amplitude and said phase is adapted to vary, such that a profile of said ultrasound energy emitted from said transducer array is amended.

7. A transducer array according to claim 6, wherein amendment of said profile of said ultrasound energy emitted from said transducer array changes a mutual relationship between a main lobes and side lobes of said profile.

8. A transducer array according to claim 7, wherein, for a given sweep range, the mutual relationship between the main lobes and the side lobes of said propagation is controlled by changing said amplitude as a function of a position of said ultrasound energy emitted from said transducer array in said sweep range.

9. A transducer array according to claim 6, wherein said control of the focal position enables an increase in a target volume that said ultrasound emission can treat without motion of said transducer array.

10. A transducer array according to claim 6, wherein said control of the focal position increases an accuracy of the focal position of said ultrasound emission, such that impingement on undesired regions is reduced.

11. A transducer array according to claim 1, wherein said at least one of said amplitude and phase is adapted to vary so as to generate, within a target area, at least two focused regions from different regions of said array.

12. A transducer array according to claim 11, wherein said at least two focused regions are directed to fall essentially on a same position within said target area such that an intensity of said ultrasound in said target area is increased.

13. A transducer array according to claim 11, wherein said at least two focused regions are directed to fall close to each other within said target area such that a volume of said target area is increased.

14. A transducer array according to claim 1, wherein said at least one of the amplitude and phase is adapted to vary so as to control a type of interaction of said ultrasound energy on a tissue of a subject.

15. A transducer array comprising:
at least one unitary element of piezoelectric material operative as a plurality of individual transducer segments by virtue of a plurality of electrode elements, said plurality of electrode elements being formed as a segmented conductive layer on at least one surface of said at least one unitary element of piezoelectric material, each segment of said conductive layer defining an individual transducer segment; and
driving circuitry for supplying high frequency voltages to at least some of said electrode elements, such that said individual transducer segments associated with said at least some electrode elements emit ultrasound energy,
wherein said driving circuitry varies at least one of an amplitude and a phase of said high frequency voltages applied to different ones of said at least some electrode elements, so as to affect propagation of said emitted ultrasound energy.

16. A transducer array according to claim 15, wherein a shift is applied to the phase of said high frequency voltages applied to different ones of said at least some electrode elements, such that said ultrasound energy emitted from said transducer array is directed at an angle in accordance with said shift of said phase.

17. A transducer array according to claim 16, wherein said shift of said phase between said high frequency voltages applied to different ones of said at least some electrode elements are varied as a function of time, such that said ultrasound energy executes a sweeping action in accordance with said variation of said shift of said phase.

18. A transducer array according to claim 16, wherein at least one of said amplitude and said phase of said high frequency voltages applied to different ones of said at least some electrode elements is varied such that a profile of said ultrasound energy emitted from said transducer array is amended.

19. A transducer array according to claim 16, wherein at least one of said amplitude and said phase of said high frequency voltages applied between different ones of said at least some electrode elements is varied such that a position of focus of said ultrasound energy emitted from said transducer array is controlled.

20. A transducer array according to claim 18, wherein amendment of said profile of said ultrasound energy emitted from said transducer array changes a mutual relationship between main lobes and side lobes of said profile.

21. A transducer array according to claim 20, wherein, for a given sweep range, the mutual relationship between the main lobes and side lobes of said profile is controlled by changing said amplitude and/or phase of said high frequency voltages as a function of the position of said emitted ultrasound energy in said sweep range.

22. A transducer array according to claim 19, wherein said control of the position of focus enables an increase in a target volume that said ultrasound emission can treat without motion of said transducer array.

23. A transducer array according to claim 19, wherein said control of the position of focus increases an accuracy of the focal position of said ultrasound emission, such that impingement on undesired regions is reduced.

24. A transducer array according to claim 15, wherein said at least one of said amplitude and phase applied to different ones of said at least some electrode elements is varied so as to generate within a target area at least two focused regions from different regions of said array.

25. A transducer array according to claim 24, wherein said at least two focused regions are directed to fall essentially on the same position within said target area such that an intensity of said ultrasound in said target area is increased.

26. A transducer array according to claim 24, wherein said at least two focused regions are directed to fall close to each other within said target area such that a volume of said target area is increased.

27. A transducer array according to claim 15, wherein said at least one of the amplitude and phase of said high frequency voltages applied to different ones of said at least some electrode elements is varied so as to control the type of interaction of said ultrasound energy on a tissue of a subject.

28. A method of generating ultrasound energy, comprising:
providing at least one unitary element of piezoelectric material having conductive layers on its first and second surfaces, at least one of said conductive layers being a segmented layer comprising a plurality of electrode elements, each of said electrode elements defining a segmental transducer;
exciting at least some of said electrode elements with high frequency voltages such that their associated segmental transducers emit ultrasound energy; and
varying at least one of the amplitude and phase of said high frequency voltages applied to different ones of at least some of said electrode elements, so as to influence a propagation of said ultrasound energy emitted from said transducer array.

29. A method of generating ultrasound energy according to claim 28, wherein a phase shift applied to different ones of said at least some electrode elements is varied as a function of time, such that said ultrasound energy executes a sweep in accordance with the variation of said phase shift.

30. A method of generating ultrasound energy according to claim 28, wherein at least one of said amplitude and said phase of said high frequency voltages applied to different ones of said at least some electrode elements is varied, such that a profile of said ultrasound energy emitted from said transducer array is amended.

31. A method of generating ultrasound energy according to claim 28, wherein at least one of said amplitude and said phase of said high frequency voltages applied to different ones of said at least some electrode elements is varied such that a position of focus of said ultrasound energy emitted from said transducer array is controlled.

32. A method of generating ultrasound energy according to claim 30, wherein amendment of said profile of said ultrasound energy emitted from said transducer array changes a mutual relationship between main lobes and side lobes of said profile.

33. A method of generating ultrasound energy according to claim 32, wherein, for a given sweep range, the mutual relationship between the main lobes and side lobes of said profile is controlled by changing said amplitude of said high frequency voltages as a function of the position of said emitted ultrasound energy in said sweep range.

34. A method of generating ultrasound energy according to claim 31, wherein said control of the position of focus enables an increase in a target volume that said ultrasound emission can treat without motion of said transducer array.

35. A method of generating ultrasound energy according to claim 31, wherein said control of the position of focus increases accuracy of the focal position of said ultrasound emission, such that impingement on undesired regions is reduced.

36. A method of generating ultrasound energy according to claim 28, wherein said at least one of said amplitude and phase applied to different ones of said at least some electrode elements is varied so as to generate within a target area at least two focused regions from different regions of said array.

37. A method of generating ultrasound energy according to claim 36, wherein said at least two focused regions are directed to fall essentially on the same position within said target area such that intensity of said ultrasound in said target area is increased.

38. A method of generating ultrasound energy according to claim 36, wherein said at least two focused regions are directed to fall close to each other within said target area such that the volume of said target area is increased.

39. A method of generating ultrasound energy according to claim 28, wherein said at least one of the amplitude and phase of said high frequency voltages applied to different ones of said at least some electrode elements is varied so as to control the type of interaction of said ultrasound energy on a tissue of a subject.

40. A method of generating ultrasound energy, comprising:
providing at least one unitary element of piezoelectric material operative as a plurality of individual transducer segments by exciting a plurality of electrode elements, said plurality of electrode elements being formed as a segmented conductive layer on a surface of said at least one unitary element of piezoelectric material, each segment of said conductive layer defining an individual transducer segment;
applying high frequency voltages to at least some of said electrode elements, such that said individual transducer segments associated with said at least some electrode elements emit ultrasound energy; and
varying at least one of the amplitude and phase of said high frequency voltages applied to different ones of said at least some electrode elements so as to affect a propagation of said emitted ultrasound energy.

41. A method of generating ultrasound energy according to claim 40, wherein said phase of said high frequency voltages applied to different ones of said at least some electrode elements is shifted such that said ultrasound energy emitted from said transducer array is directed at an angle in accordance with the phase shift.

42. A method of generating ultrasound energy according to claim 40, wherein the phase shift between said high frequency voltages applied to different ones of said at least some electrode elements is varied as a function of time, such that said ultrasound energy executes a sweep in accordance with said variation of said phase shift.

43. A method of generating ultrasound energy according to claim 40, wherein at least one of said amplitude and said phase of said high frequency voltages applied to different ones of said at least some electrode elements is varied, such that a profile of said ultrasound energy emitted from said transducer array is amended.

44. A method of generating ultrasound energy according to claim 40, wherein at least one of said amplitude and said phase of said high frequency voltages applied to different ones of said at least some electrode elements is varied such that a position of focus of said ultrasound energy emitted from said transducer array is controlled.

45. A method of moving ultrasound energy through a target volume, comprising:
providing at least one unitary element of piezoelectric material having conductive layers on its surfaces, at least one of said conductive layers being a segmented layer comprising a plurality of electrode elements, each of said electrode elements defining a segmental transducer;
positioning said at least one unitary element of piezoelectric material in proximity to said target area;
exciting at least some of said electrode elements with high frequency voltages such that their associated segmental transducer emit ultrasound energy; and
varying the phase of said high frequency voltages applied to different ones of at least some of said electrode elements such that said ultrasound moves through said target volume.
